(19) United States
(12) Patent Application Publication
(10) Pub. No.: US 2026/0284396 A1
(43) Pub. Date: Sep. 24, 2026

(54) CLOSED-LOOP NEUROMODULATION TO TREAT A CONDITION OF A BRAIN USING AN ADAPTIVE BRAIN STATE MODEL

(71) Applicant: VANDERBILT UNIVERSITY, Nashville, TN (US)

(72) Inventors: Graham JOHNSON, Nashville, TN (US); Dario Englot, Nashville, TN (US)

(21) Appl. No.: 19/689,569

(22) Filed: May 27, 2026

Related U.S. Application Data
(63) Continuation-in-part of application No. PCT/US2025/031208, filed on May 28, 2025.
(60) Provisional application No. 63/813,532, filed on May 28, 2025.

(51) Int. Cl. A61N 1/36

(52) U.S. Cl. CPC A61N 1/36064; A61N 1/36139

(57) ABSTRACT

A condition of a brain of a previously unseen patient can be treated with closed-loop neuromodulation using a trained brain state model constructed by capturing blended brain-states across a plurality of patients. At least one recording electrode can record conduction data from at least a portion of the brain. At least one stimulating electrode can apply an electrical signal to another portion of the brain. A controller can execute stored instructions and a stored patient-specific model to: receive the conduction data at a time; project the conduction data through a trained brain state model to determine a brain state, wherein the trained brain state model enables brain-state trajectory prediction of the previously unseen patient; and update at least one parameter of an electrical signal based on a propensity of the brain state at the time to cause an effect of the condition of the brain.

### RELATED APPLICATIONS

This application claims priority to U.S. Provisional Application Ser. No. 63/813,532, filed May 28, 2025, entitled “CLOSED-LOOP NEUROMODULATION TO TREAT A CONDITION OF A BRAIN USING AN ADAPTIVE STATE MODEL”.

This application is also a bypass Continuation-in-Part (CIP) of International Application No. PCT/US2025/031208, filed 28 May 2025, which claims priority to U.S. application Ser. No. 18/676,682, filed May 29, 2024, entitled “CLOSED-LOOP NEUROMODULATION TO TREAT A CONDITION OF A BRAIN USING AN ADAPTIVE STATE MODEL”.

The entirety of these applications is hereby incorporated by reference for all purposes.

### GOVERNMENT SUPPORT

This invention was made with government support under NS112252 awarded by the National Institutes of Health. The government has certain rights in the invention.

### TECHNICAL FIELD

The present disclosure relates generally to treating a condition of a brain and, more specifically, to systems and methods that capture blended brain-states across humans and enable generalized neurostimulation of previously unseen patients (the previously unseen patients are not included in the data used to train the brain-state model).

### BACKGROUND

Epilepsy is a chronic brain condition that affects around 50 million people worldwide and causes recurring seizures. Drug-resistant epilepsy (DRE) is a subset of epilepsy where patients do not successfully respond to pharmaceutical therapy and instead rely on other techniques, such as neuromodulation therapies, to treat the DRE. Neuromodulation therapies have advanced considerably over the last fifteen years, such that now the brain can be monitored for one or more biomarker(s) of active disease states (that are constant) and at least a portion of the brain can be stimulated when at least one of the biomarker(s) is detected. Now one treatment for DRE is seizure forecasting with responsive neurostimulation (RNS). Seizure forecasting monitors for a pre-ictal signature(s), which may include a plurality of constant biomarkers, that appears immediately preceding a seizure (a short time scale biomarker) and RNS then delivers a high-energy stimulation upon detection of the pre-ictal signature(s) in an attempt to halt seizure progression. Evidence has emerged, however, that the short timescale biomarker (e.g., the pre-ictal signature(s)) and the responsive high energy stimulation may not be the most effective means to halt and/or prevent seizures.

### SUMMARY

Described herein are systems and methods that apply neuromodulation (e.g., in a closed-loop) to treat a condition of the brain by implementing and adapting a trained patient-specific model of brain states to adaptively determine a brain state at a time and determine if that brain state should trigger treatment and/or a change to a current treatment. The adaptability of the trained patient-specific model is advantageous over traditional, stagnant biomarkers at least because the trained patient-specific model can be continuously and automatically updated.

In an aspect, the present disclosure can include a system that can be used for closed-loop neuromodulation to treat a condition of the brain. The system can include at least one recording electrode configured to record conduction data from at least a portion of the brain. The system can also include at least one stimulating electrode configured to apply an electrical signal, generated and configured by a generator, to at least another portion of the brain. The electrical signal comprises at least one parameter. The system can also include a controller in electrical communication with the at least one recording electrode and the generator. The controller comprises a non-transitory memory configured to store instructions and a trained patient-specific model and a processor configured to execute the instructions and the trained patient-specific model to: receive the conduction data at a time; project the conduction data through the trained patient-specific model to determine a brain state at the time; update the at least one parameter of the electrical signal based on a propensity of the brain state at the time to cause an effect of the condition of the brain; and update the trained patient-specific model to include the brain state at the time and an effect of the updated at least one parameter of the electrical signal on the brain state at the time.

In another aspect, the present disclosure can include a method for closed-loop neuromodulation to treat a condition of the brain. The method can include: receiving, by a system comprising a processor, conduction data at a time from at least one recording electrode in communication with the processor, wherein the at least one recording electrode records conduction data from at least a portion of the brain; projecting, by the system, the conduction data at the time through a trained patient-specific model to determine a brain state at the time; updating, by the system, at least one parameter of an electrical signal based on a propensity of the brain state at the time to cause an effect of the condition of the brain, wherein the processor is further in communication with at least a generator that generates the electrical signal and provides the electrical signal to at least one stimulation electrode that applies the electrical signal to at least another portion of the brain; and updating, by the system, the trained patient-specific model to include the brain state at the time and an effect of the application of the updated the at least one parameter of the electrical signal on the brain state at the time.

Also described herein are systems and methods that capture blended brain-states across humans and enable generalized neurostimulation of previously unseen patients (not included in the data used to train the brain-state model).

In an aspect, a method for closed-loop neuromodulation to treat a condition of a brain of a previously unseen patient is described. The method can be performed by a system comprising a processor. The method can include: receiving conduction data at a time from a plurality of recording electrodes in communication with the processor, wherein the plurality of recording electrode record conduction data from at least a portion of the brain of the previously unseen patient. After receiving the conduction data, the conduction data at the time can be projected through a trained brain state model to determine a brain state of the previously unseen patient at the time, wherein the trained brain state model is constructed without training data from the previously unseen patient, wherein the trained brain state model is constructed by capturing blended brain-states across a plurality of patients and enables brain-state trajectory prediction for the previously unseen patient. At least one parameter of an electrical signal can be updated based on a propensity of the brain state of the previously unseen patient at the time to cause an effect of the condition of the brain. Based on the propensity of the brain state, at least a generator generates the electrical signal and provides the electrical signal to at least one stimulation device that applies the electrical signal to at least another portion of the brain, wherein the at least one parameter and the effect of the condition are based on the blended brain-states across the plurality of patients and the conduction data at the time.

In another aspect, a system can include: a plurality of recording electrodes configured to record conduction from at least a portion of a brain of a previously unseen patient; an output device; and at least one controller, in communication with the plurality of electrodes and the display device. Each controller can include a memory configured to store a trained brain state model and instructions, wherein the trained brain state model comprises a brain state embedder, a brain state predictor, and a brain state visualizer, and wherein the trained brain state model was not trained on the conduction data of the previously unseen patient, and a processor configured to execute at least a portion of the instructions. Upon execution of at least the portion of the instructions, the controller can: receive by the brain state embedder, the conduction data from the plurality of recording electrodes; embed, by the brain state embedder, the conduction data into a common latent space with a number of dimensions, wherein the brain state embedder forms outputs comprising at least embedded conduction data in the common latent space; predict, by the brain state predictor, at least one predicted future embedded conduction data in the common latent space; reduce and organize, by the brain state visualizer, the conduction data in the common latent space and the at least one predicted future embedded conduction data in the common latent space into two dimension; and visualize, by the brain state visualizer via the output device, at least one two dimensional (2D) topology map of a current brain state of the previously unseen patient and/or a predicted future brain state of the previously unseen patient.

In a further aspect, a method for training a brain state model to reconstruct conduction data from a previously unseen patient and encode brain-state embeddings into clinically meaningful clusters within a latent space is described. The method can be performed by a system comprising a processor. The method comprising: receiving raw conduction data of a brain from a plurality of patients; transforming the raw conduction data into a 1024 dimensional vector; training a Gaussian-mixture variational auto-encoder (GM-VAE) with an adversarial estimation of Kullback-Leibler divergence; and outputting a clustered, smooth latent space of blended brain states across patients; and training a brain state predictor on embeddings of the clustered, smooth latent space of the blended brain states of the plurality of patients to predict future brain states.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram of a system for closed-loop neuromodulation to treat a condition of a brain;

FIG. 2 is an example of the controller of FIG. 1;

FIG. 3 is an example of training a patient-specific model of FIG. 2;

FIG. 4 is an example of executing the trained patient-specific model of FIG. 2;

FIG. 5 is a process flow diagram of a method for treating a condition of the brain using closed-loop neuromodulation;

FIG. 6 shows an example of synergistic adaptable closed-loop neuromodels to elucidate brain-state and deliver brain-state modulating low-energy stimulation;

FIG. 7 shows an example of a brain-state modeling process;

FIG. 8 shows an example of a custom asymmetric recurrent beta-variational autoencoder architecture;

FIG. 9 shows an example time-shift invariant loss function for multi-channel timeseries forecasting;

FIG. 10 shows an example Kullback-Leibler (KL) and Learning Rate (LR) annealing schedules;

FIG. 11 shows an example latent dimensionality reduction and spatial clustering;

FIG. 12 shows example latent spaces depicting clustered functional brain-states;

FIG. 13 shows experimental results of significant perturbation by brain-states by single-pulse electrical stimulation;

FIGS. 14-16 show training and validation of latent spaces for different subjects;

FIG. 17 shows an example of Subject 2's seizure propensity timeline with vertical lines indicating an ictal event;

FIG. 18 is an example of training a trained brain state model of FIG. 2;

FIG. 19 is a process flow diagram of a method for treating a previously unseen patient using a trained brain state model;

FIG. 20 is a process flow diagram of a method for treating a condition in a previously unseen patient;

FIG. 21 is a process flow diagram of a method for visualizing at least one 2D topology map of a current brain state and a predicted future brain state of a previously unseen patient;

FIG. 22 is a process flow diagram of a method for training a brain state predictor to predict brain states and/or future brain states of a previously unseen patient;

FIG. 23 shows an example overview of the Brain-State Foundation paradigm of the brain state model (BSM)/brain foundation model (BFM);

FIG. 24 shows examples of how data flows through the model and results of generalized raw signal reconstruction;

FIG. 25 shows an example detailed architecture of the BSM;

FIG. 26 shows an example post-hoc interpretation of the BFM embedding space;

FIG. 27 shows example sleep stages that generalize to unseen subject recordings;

FIG. 28 shows that the BFM is able to accurately predict brain-state trajectories;

FIG. 29 shows a custom patient-specific model for brain-state embedding used as a steppingstone for creating the generalized brain state model;

FIG. 30 shows examples of manifold reduction of patient-specific brain-states for three different subjects;

FIG. 31 shows examples of using brain-state clusters to predict experiencing a seizure; and

FIG. 32 shows examples of low-energy single-pulse electrical stimulation (SPES) effects on brain states.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an”, and “the” can also include the plural forms unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

As used herein, the term “condition of a patient's brain” can refer to any neurological pathology, including injury, illness, or disorder, that affects the brain. Specific, but not limiting, examples of conditions of the brain can include drug-resistant epilepsy (DRE), obsessive compulsive disorder, Tourette's syndrome, major depressive disorder, schizophrenia, bipolar disorder, binge eating disorder, substance abuse disorder, or the like.

As used herein, the term “epilepsy” can refer to a neurological pathology associated with abnormal electrical activity in the brain in which a patient has two or more unprovoked seizures that occur more than 24 hours apart.

As used herein, the term “seizure” can refer to a sudden, uncontrolled burst of electrical activity in the brain. A seizure can cause changes in behavior, movements, feeling, levels of consciousness, or the like.

As used herein, the term “drug resistant epilepsy”, or “DRE”, can refer to a type of epilepsy where seizures do not successfully respond to medication therapy (e.g., at least two antiseizure medications). DRE may also be referred to as intractable, medically refractory, pharmacoresistant, or the like.

As used herein, the term “neuromodulation” can refer to the alteration of neural activity through targeted delivery of a stimulus, such as one or more of electrical stimulation, application of chemical agents, or the like.

As used herein, the term “low energy” can refer to a characterization of electrical stimulation treatment having current levels of 1 milliamps-5 milliamps and a continuous stimulation frequency of 10 Hz or less or an intermittent stimulation with frequency of greater than 10 Hz for less than 1 second followed by at least 1 second of no stimulation.

As used herein, the term “electrode” can refer to a solid electrical conductor that carries electric current into one or more non-metallic elements (e.g., within a patient's body.). Electrodes can record data and/or deliver electrical stimulation and can be internal electrodes (e.g., intracranial electrodes) and/or surface electrodes. Electrodes configured to record data from a patient can be referred to as recording electrodes. Electrodes configured to stimulate a patient (e.g., with an electrical signal) can be referred to as stimulating electrodes. It should be understood that recording electrodes and stimulating electrodes can be within the same electrode and/or can be within separate electrodes.

As used herein, the term “conduction data” can refer to electrical activity (e.g., one or more signals) recorded from a patient's brain by one or more electrodes. For example, the conduction data can be recorded using electroencephalography (also referred to as EEG), a test that measures electrical activity a patient's brain. Electrodes used for EEG can be external, internal, or the like. One example of EEG is intracranial EEG (also referred to as iEEG) where EEG is obtained with intracranial electrodes, an example of which is stereoelectroencephalography (also referred to as SEEG). Another example of EEG is electrocorticography (ECOG), where EEG is acquired by strips or grids of electrodes implanted over the bare cortex in subdural space. A further example of EEG uses electrodes attached to the scalp.

As used herein, the term “latent space”, also referred to as “latent feature space” and “embedding space”, can refer to a multi-dimensional space that encodes a meaningful representation of characteristics of a set of data (e.g., embedded within a manifold in which items resembling each other are positioned closer to one another). The latent space can be high-dimensional, complex, and non-linear. The latent space provides a compressed understanding to a computer through a spatial representation.

As used herein, the term “brain state”, also referred to as “brain-state”, can refer to a representation of electrical activity in one or more areas of the brain at a given time.

As used herein, the term “patient”, also referred to as subject and other similar terms, can refer to any warm-blooded organism, including, but not limited to, a human being, a pig, a rat, a mouse, a dog, a cat, a goat, a sheep, a horse, a monkey, an ape, a rabbit, a cow, etc.

As used herein, the term “previously unseen patient” can refer to a patient, conduction data taken from the patient and analyzed by a brain state model, and/or one or more brain states determined by the brain state model from the conduction data taken from the patient. Previously unseen references that the brain state model was not trained with data (e.g., conduction data) taken from the patient. In other words, that the brain state model was not customized to the patient previously, has never seen the patient's conduction data previously, determined a brain state for the patient previously, or predicted a brain state of the patient previously.

### II. Overview

Neuromodulation for drug resistant epilepsy (DRE) (and other neurological pathologies) has advanced considerably in recent years and has made closed-loop neuromodulation possible. The brain can be monitored for potential biomarkers of active disease states and stimulation can be applied based on a feedback loop for the biomarkers. In fact, Responsive Neurostimulation (RNS) is used for patients with DRE to detect immediate pre-ictal signatures and deliver high-energy stimulation in an attempt to abort seizure progression. However, recent research has shown that RNS based on pre-ictal signatures may be too late to effectively halt seizures and that low-energy stimulation may be more effective (this is in alignment with clinical observations that seizures tend to occur on long-range periodic timescales).

As such, described herein are systems and methods that can neuromodulate a patient's brain to treat a condition of the brain (including DRE) based on an adaptable trained patient-specific model of brain states (Invention 1) or a generalized trained brain state model (Invention 2) significantly before current pre-ictal signatures (e.g., on a long timescale).

Indeed, the trained patient-specific model can be continuously and automatically updated as conduction data of the patient is recorded to further personalize and improve the treatment. Currently monitored biomarkers struggle to accurately forecast seizures in many instances, which may be at least partially due to their being unidimensional, as well as having too short a time scale. The trained patient-specific model can be multi-dimensional, accounting for many dimensions of data in determining and identifying the vast quantity of possible brain states and their long-term consequences on a given patient. The systems and methods described herein utilize novel signal processing and normalization, a custom asymmetric variational autoencoder, and a novel loss paradigm to elucidate a trained patient-specific model of brain-state(s) that can further be trained to deliver proper neurostimulation (e.g., low energy) over long time periods to maintain satisfactory brain states of a patient. Brain state at a given time can be quantified on a gradient scale from low propensity to high propensity (e.g., for a seizure or other effect of a condition of the brain) in a method similar to a forecasting a tornado (e.g., no alert, watch, warning). It should be noted that the trained patient-specific model can be nearly infinitely multi-dimensional (e.g., 512 dimensions or more) well beyond the limits of human comprehension and unaided computations.

The generalized brain state model can be utilized on patient's that have not been previously used as part of the model's training data-allowing for quicker diagnosis and/or treatment. The goal of the generalized brain state model is to have a paradigm to elucidate highly-abstracted brain-states in a framework that generalizes to unseen subjects. Specifically, the generalized brain state model can utilize a fundamental relationship between brain activity across humans to instantaneously capture a new subject's clinically relevant brain-state and accurately predict the future brain-state trajectory. From this point, the model can further apply neuromodulation to alter the current and/or predicted further brain state trajectory. To accomplish this, the generalized brain state model includes a machine learning model trained on a broad range of data in a self-supervised manner that can be adapted to downstream tasks. In this context, the current application presents a generalized brain state model trained on intracranial electroencephalography (iEEG) data from people with drug-resistant epilepsy (DRE) as the first example of a generalizable brain-state model, coined Brain-state Foundation Model (BFM) or Brain State Model (BSM). The BFM/BSM can also be used, as an example, for treatment of epilepsy in adaptive closed loop neuromodulation.

### Invention 1—Creating and Using a Patient-Specific Customized Brain-State Model

### III. Systems 1

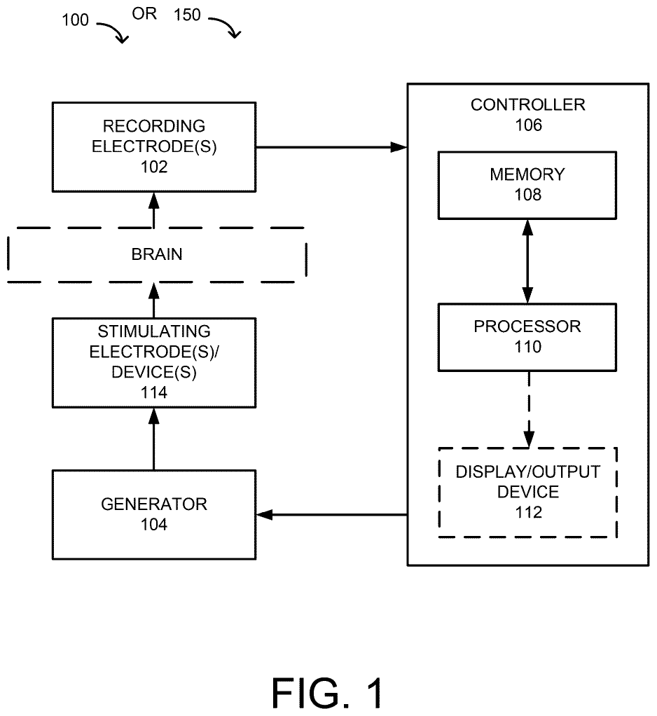

An aspect of the present disclosure can include a system 100 (FIG. 1) that can treat a condition of a patient's brain using neuromodulation (e.g., closed-loop neuromodulation). The condition of the patient's brain can refer to any neurological pathology, including injury, illness, or disorder, that affects the brain. Specific examples of conditions of the brain can include one or more of epilepsy, including drug-resistant epilepsy (DRE), obsessive compulsive disorder, Tourette's syndrome, major depressive disorder, schizophrenia, bipolar disorder, binge eating disorder, substance abuse disorder, or the like. For instance, neuromodulation with the system 100 can prevent and/or control seizures, obsessions and/or compulsions, tics, depressive episodes, schizophrenic symptoms, bipolar swings, binging or substance compulsions, or the like. Alternatively, the system 100 can be used in applications that use conduction in the brain to perform a function, such as BCI (brain-computer interfaces). Generally, neuromodulation is the alteration of neural activity through targeted delivery of a stimulus (e.g., electrical, chemical, magnetic, radiation (e.g., heat, light, or the like), etc.). The neuromodulation, in some instances, can be electrical neuromodulation that can stimulate one or more portions of the brain with a low energy electrical signal over long time periods. It should be understood that while only electrical neuromodulation is described in detail, magnetic stimulation (as well as other therapies traditionally used to treat neurological conditions) is also contemplated as able to work in a similar manner, and only electrical modulation is referred to herein solely for ease of description.

As shown in FIG. 1, the system 100 can include one or more recording electrodes (recording electrode(s) 102) that can record conduction information from at least a portion of the patient's brain (shown in FIG. 1 as the dashed box labeled brain). Conduction data can refer to electrical activity recorded from the patient's brain. For example, the conduction data can be recorded using electroencephalography (also referred to as EEG), a test that measures electrical activity a patient's brain. Recording electrode(s) 102 can be at least one of external, internal, intracranial, or the like. For instance, the recording electrode(s) 102 can include one or more intracranial electrodes for intracranial EEG (also referred to as iEEG) or stereoelectroencephalography (also referred to as SEEG). In another instance, the recording electrode(s) 102 can be one or more electrodes implanted over the cortex in subdural space for electrocorticography (ECoG). In a further instance the recording electrode(s) 102 can be scalp electrodes configured for scalp EEG recording. In each case, the conduction data can provide a representation of electrical activity in one or more areas of the brain at a given time. The recording electrode(s) 102 can be any number N that is one or greater.

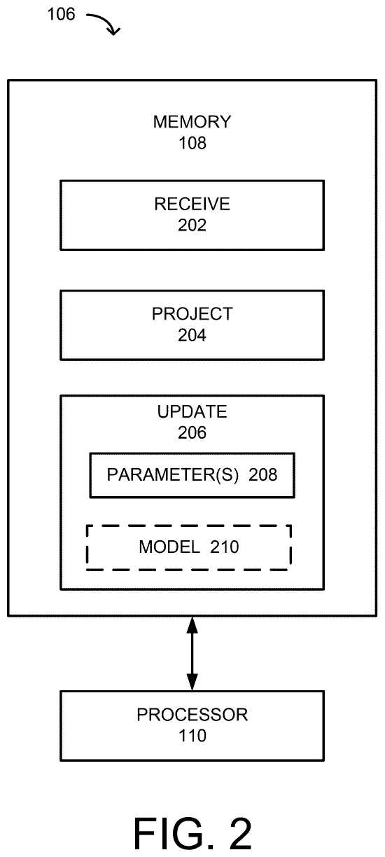

Conduction data recorded by the recording electrode(s) 102 (N channels of data corresponding to the N recording electrodes 102) can be sent to controller 106. The conduction data can be recorded and transmitted at one or more frequencies. The recording electrode(s) 102 and the controller 106 can be in wired and/or wireless electrical communication. The controller 106 can include memory 108 (which is non-transitory) and a processor 110 (e.g., a microprocessor, a computing device, a state machine, a signal processing chip, or the like). In some instances, at least a portion of the memory 108 and processor 110 can be embedded within the same device (e.g., a microprocessor). In other instances, the memory 108 and the processor 110 can be entirely separate devices. The memory 108 can store instructions related to training and execution of a trained patient-specific model and the trained patient-specific model itself. The processor 110 can execute the instructions and the trained patient-specific model, for instance, to determine brain states from the conduction data and whether, and which one or more parameters of the electrical neuromodulation, to update the neuromodulation treating the condition of the brain (more detail shown in FIG. 2). The controller 106 may also, in some instances, have a display 112 (or other type of output device for visual, audible, or tactile output) and/or an input device (not shown, for inputting one or more instructions, limits, additional data, or the like).

Upon determining that at least a portion of the conduction data at a time corresponds to a brain state indicating a need for neuromodulation and/or a change in neuromodulation (e.g., a high propensity brain state, described in further detail below), the controller 106 can determine (if no neuromodulation is being applied) or update (if neuromodulation is already being applied) one or more parameters of an electrical signal (e.g., corresponding to shape, current, voltage, amplitude, frequency, pulsation, timing, etc.) and send the one or more new or updated parameters to a generator 104. The generator 104 may be a standalone device in electrical communication (wired and/or wireless) with the controller 106 and the stimulating electrode(s) 114 (as shown), part of the controller 106, part of a system including the stimulating electrode(s) 114, etc.). The generator 104 can receive the one or more new or updated parameters and create or update the electrical signal based on the new or updated parameters. The electrical signal can be sent to one or more stimulating electrodes (stimulating electrode(s) 114) for application of the configured electrical signal to at least another portion of the brain for neuromodulation. The at least the other portion of the brain can include at least a portion of the same portion of the brain monitored by the recording electrode(s) 102, different from the portion of the brain monitored by the recording electrode(s), and/or at least partially the same and at least partially different. The stimulating electrode(s) 114 can be internal electrodes (e.g., intracranial electrodes) and/or surface electrodes (e.g., positioned on the scalp). In some instances, the recording electrode(s) 102 and the stimulating electrode(s) 114 can be the same electrodes. In other instances, the recording electrode(s) 102 and the stimulating electrode(s) 114 can be unique and distinct from one another.

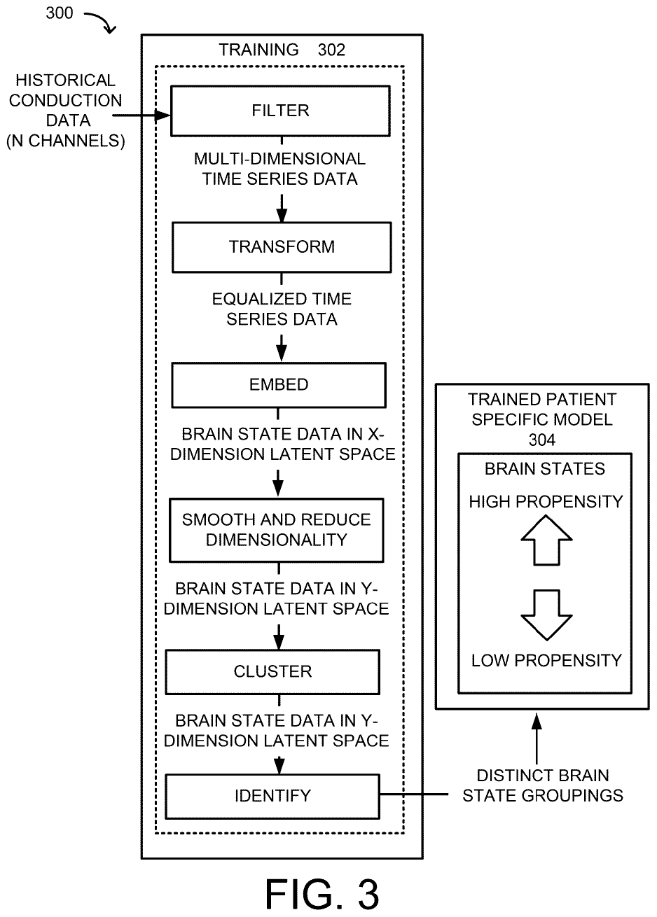

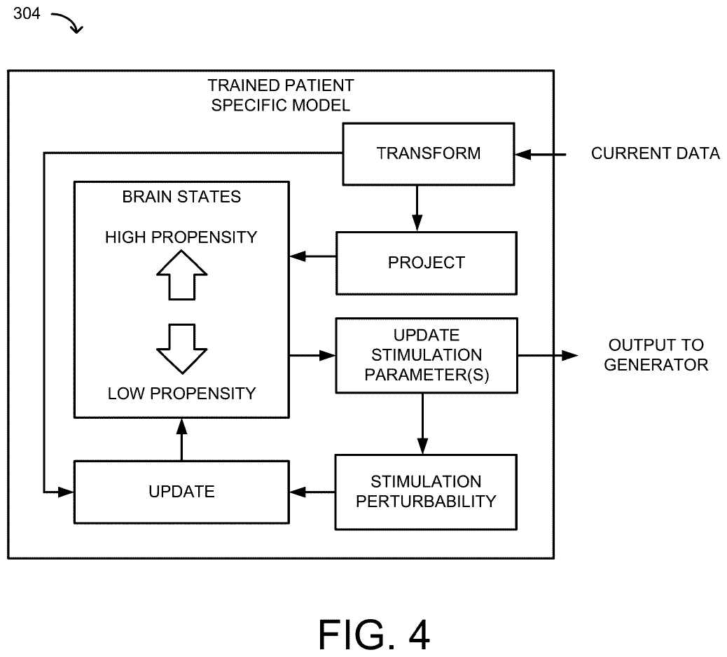

The system 100 can be a closed-loop between the recording electrode(s) 102, the controller 106, and the generator 104/stimulating electrode(s) 114. To implement the neuromodulation in the closed-loop, the controller 106 can train, implement, and adapt the trained patient-specific model of brain states to adaptively determine a propensity of a brain state at a given time towards one or more effects or symptoms of a condition of the brain of the patient and determine whether that brain state should trigger treatment and/or a change to a current treatment. An example of the functionality of the controller 106 is shown in FIG. 2, further details are discussed with respect to FIGS. 3 and 4. Conduction data recorded by the recording electrodes can be received at a time (receive 202). The conduction data can then be projected through the trained patient-specific model to determine a brain state at the time and the brain state's propensity (project 204). For example, in use after training, the processor 110 can execute the trained patient-specific model and can process the conduction data at the time into brain state data at the time to be compatible with the trained patient-specific model. The brain state data can then be projected through the trained patient-specific model to determine the propensity of the brain state at the time and whether that brain state should trigger treatment and/or a change to a current treatment.

The trained patient-specific model can employ a multi-dimensional latent space to determine whether the brain state at the time has a propensity to cause an effect on a condition of the brain. For instance, the brain state at the time can be run through the multi-dimensional latent space and analyzed with respect to a multitude of brain states with known effects on the condition of the brain to determine the current propensity to cause an effect on the condition of the brain. It should be noted that the this is far from a simple one-to-one comparison (or even a plurality of one-to-one comparisons) but instead includes multi-dimensional comparisons of positions and calculations of a plurality of interacting signs and brain states that a person cannot accomplish in the mind. As an example, the positions can correspond to cautions similar to tornado watches and warnings. In one area, the positions can correspond to no watch (low propensity), another area can correspond to a watch if some early signs appear (mid-level propensity), while another area can correspond to a warning when more or stronger signs appear (high propensity). It should be understood that these are only examples, and that the propensity can be on a gradient that can include many different propensities that may or may not be pointed in nature. The trained patient-specific model can include historical data with known outcomes for the comparison but need not include each of the specific brain states being compared during use (e.g., the patient-specific model can predict propensity even for brain state data that it has not yet seen as it is believed that no two brain states are entirely identical in latent space). It should be further noted that, while not wishing to be bound by theory, the exact time course of brain states in latent space before a given seizure cannot be reliably labeled (e.g., will or will not result in an effect) so propensity is determined rather than categorization of brain states. When the brain state indicates a propensity to cause an effect on the condition of the brain, at least one parameter of the electrical signal for the neuromodulation (parameter(s) 208) and the trained patient specific model (model 210) can be updated (updated 206). As part of the updating the at least one parameter can be updated and sent to the generator via the processor 110 and the trained patient-specific model can be updated to include that brain state, the effect on the patient, and/or the effect of the change in the one or more parameters on the patient.

Shown in FIG. 3 an example 300 of how the trained patient specific model is trained for each unique patient. Historical conduction data (from N-channels corresponding to N recording electrodes) from a given previous time period can be input into a training 302 module. The training 302 module can receive the historical conduction data and filter the historical conduction data into multi-dimensional time series data signals for the N channels. The filtering can, for instance, include a zero-phase shift 5^{th} order Butterworth filter with passbands of 1 Hz-59 Hz, 61 Hz-119 Hz, and 121 Hz-179 Hz. The historical conduction data may also be re-sampled to the lowest sample rate of all the historical conduction data. For example, if the sampling rate of the historical conduction data ranged from 512 Hz to 2048 Hz, then the historical conduction data can all be resampled at 512 Hz. The training 302 module can then equalize the multi-dimensional time series data signal using a zero-centered one-dimensional histogram equalization (ZHE) scheme to form equalized time series data. For instance, the signal can be split into the positive and negative domains, then a 10,000-bin histogram can be filled for each domain from 0 to the absolute value of highest voltage. Then the linear transfer function from the time series signal to the equalized signal can be calculated using the cumulative distribution function for the positive and negative domain separately. The result is a signal that preserves the physiological meaning of zero, but has data evenly distributed between −1 and 1. A normalization scheme can then be applied, such as normalizing all the subject's time series to the first, for instance, 24 hours, of data to produce equalized time series data for each channel.

Then, the training 302 module can embed the equalized time series data into a multi-dimensional latent space using an asymmetric recurrent variational autoencoder (AR-βVAE) to form the brain state data in X dimension-latent data space (e.g., having 512 dimensions, but may have any number of dimensions limited only by processing power of the computer and the highest sampling rate of the conduction data) and, in some instances, data forecasting, smooth the latent data (e.g., with a 10-second averaging window and 1 second stride, but any sized-averaging window and/or stride can be used limited by the processing power of the computer). It should be noted that 512 dimensions is far beyond the range of human comprehension.

For instance, the equalized time series data can be compressed into short data epochs (e.g., 0.5 seconds, but can be any short time segment) in 512-dimensional latent space. From there the immediate future (e.g., 0.125 seconds or the like) of all channel data can be simultaneously forecast. The AR-βVAE can be architected to accept N channels of X number of samples (for instance 88-186 channels by 256 samples) and can have multiple layers. The top layer can utilize a Gated Recurrent Unit (GRU) to interface with the input data (the equalized time series data in epochs for the N channels and X samples). The GRU can learn short- and long-range signal features. The GRU can be three layers and bidirectional, with a resulting hidden dimension of 2N that can be subsampled every eight brain states. While not wishing to be bound by theory, this can help the GRU to maintain learning capacity and not be forced to forget data motifs across the forward and reversed sequence lengths. The subsampled hidden states can then be flattened and fed into the β-VAE. The β-VAE can include fully-connected layer feeding into mean and log-variance layers that can be followed by a standard noise-injection reparameterization trick to determine the latent space. The latent space can be regularized by Kulback-Leibler (KL) Divergence and set to a number Y (e.g., 512). The decoder can output an N×64 sample forecast on all channels simultaneously that are compressed into 1×Y (e.g., 512) latent dimensions, which, not wishing to be bound by theory, best embed the necessary information to forecast the next N×64 timepoints in the original input signal. A dropout ratio (e.g., 0.1, 0.2, 0.3, or the like) can be used on the middle fully connected layer of the decoder (e.g., to promote concise and meaningful latent embeddings).

The decoding portion of the β-VAE can be asymmetric, to account for this a custom loss function (called Circular Minimum Hyperbolic Cosine Loss (cMin-LogCosh)) can be applied that allows for varying time shift of the forecasted signals. For instance, the custom loss function can be based on the hyperbolic cosine loss function and the N×64 predicted values can be wrapped in a circle with a stride of one sample and the LogCosh loss calculated every stride resulting in 64 individual loss values. The minimum LogCosh loss can be determined and returned for backpropagation through the model. Additional data transformation may also be used to increase stability, such as learning rate annealing. The 512 dimensional data can also be smoothed with a 10 second averaging window and a 1 second stride to greatly increased the signal-to-noise ratio prior to the manifold approximation.

The training 302 module can then reduce the dimensionality of the smooth latent data through a pairwise controlled manifold approximation and projection (PaCMAP) (e.g., from 512 to 10), cluster the PaCMAP reduced dimensional space data (10 dimensional data, still beyond the range of human comprehension). The reduced dimensional space data can then be fed into a Hierarchical Density Based Spatial Clustering of Applications of Noise (HDBSCAN) for clustering. Distinct brain state grouping(s) for the historical data can be identified in the reduced dimension and clustered data. The historical conduction data over which the trained patient-specific model is trained can be brain-state data for a previous time period (e.g., of more than 10 minutes, 30 minutes, 1 hour or more, 1 day or more, 3 days or more, etc.). The distinct brain state groupings discovered by the training 302 module can be used to form the high propensity to low propensity designations (e.g., on a gradient) in the trained patient-specific model 304 (shown in greater detail in FIG. 4). It should be understood that the training can be done before the model is used (e.g., using historical data from the patient, historical data from patients suffering from the same general condition, etc.) and can be continued for each “update” during implementation of the model for closed-loop neuromodulation.

Referring now to FIG. 4, illustrated is an example of using the trained patient specific model 304 in greater detail. The trained patient specific model 304 can be trained as shown in FIG. 3. Then new conduction data (recorded by the recording electrodes) can be input into trained patient specific model. The new conduction data can undergo the same transformation steps as described above with respect to the historical conduction data in FIG. 3 (e.g., take the conduction data and process and filter the conduction data into multi-dimensional time series data for the N channels, equalize the multi-dimensional time series date using a zero-centered one-dimensional histogram equalization (ZHE) to form equalized time series data, embed the equalized time series data into a multi-dimensional latent space using an asymmetric recurrent variational autoencoder (AR-βVAE) to form the brain state data in X dimension-latent data space (e.g., 512 dimensions) and, in some instances, data forecasting, smooth the X-dimensional latent data (e.g., with a 10-second averaging window and 1 second stride), reduce the dimensionality of the data through a pairwise controlled manifold approximation and projection (PaCMAP) (e.g., from 512 to 10), cluster the PaCMAP reduced dimensional space (10 dimensional data) with a Hierarchical Density Based Spatial Clustering of Applications of Noise (HDBSCAN); new distinct brain state grouping(s) can be identified in the reduced dimension and clustered data). The new distinct brain state groupings for the time can be projected through the trained patient-specific model and analyze with respect to previously identified groupings (from the training). A propensity can be assigned to the brain state groupings at the time and stimulation parameters can be updated based on the propensity and sent to the generator. Stimulation perturbability can be determined and the determination can be then fed back into the training 302 module with the current conduction data (that may be processed) for further updating the model (e.g., to make the model better with more current historical data).

### IV. Methods 1

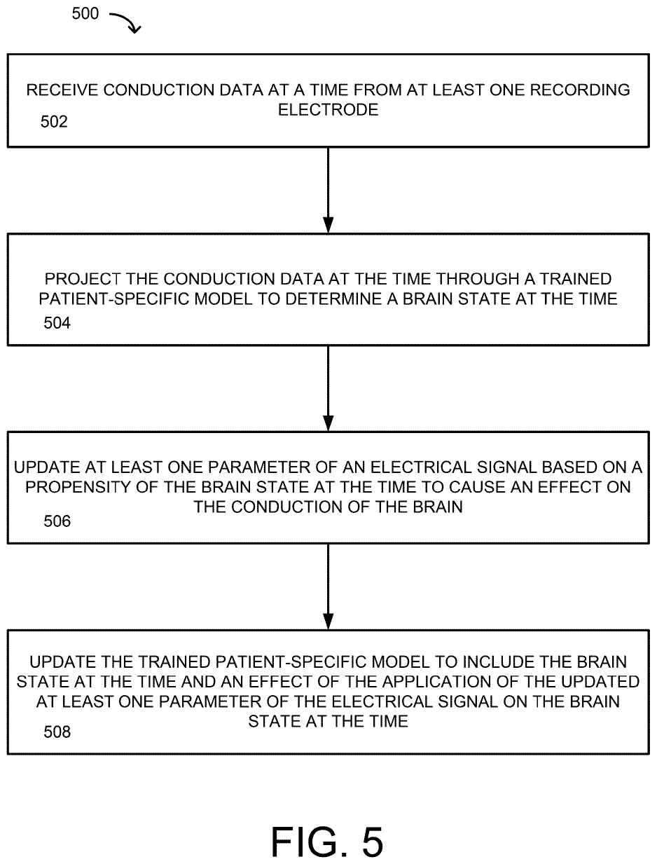

Another aspect of the present disclosure can include method 500 (FIG. 5) for treating a condition of the brain using closed-loop neuromodulation. The method 500 can be executed using the system 100 (shown in FIG. 1 and modified by FIGS. 2-4). It should be understood that system 100 includes one or more recording electrode(s) that record conduction data from an area of at least part of the brain, a controller that receives the conduction data, implements a model of brain state to process the conduction data and determine a likelihood of the brain state contributing to a brain condition, and (if necessary) output a change of one or more parameters of a stimulation, a generator that receives the output and changes the one or more parameters and generates the stimulation, and one or more stimulating electrode(s) to deliver the stimulation to another at least part of the brain (which may be the same and/or different than where the conduction data is recorded).

For purposes of simplicity, the method 500 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 500, nor is method 500 limited to the illustrated aspects.

The method 500 illustrates the actions of the controller to performed closed-loop neuromodulation to treat a condition of the brain. It will be understood that the controller can have a non-transitory memory and a processor. The non-transitory memory can store the instructions of method 500, as well as a patient-specific model (e.g., trained for a certain patient). The processor can access the memory and execute the instructions with the trained patient-specific model. Each of the steps of method 500 can be executed by the processor of the controller of system 100, for example. The method 500 can continuously modify the neuromodulation in a closed-loop to treat the condition of the patient's brain. The condition of the patient's brain can be a neurological pathology, including injury, illness, or disorder, that affects the brain. Specific examples of conditions of the brain can include one or more of epilepsy, such as drug-resistant epilepsy (DRE), obsessive compulsive disorder, Tourette's syndrome, major depressive disorder, schizophrenia, bipolar disorder, binge eating disorder, substance abuse disorder, or the like. The neuromodulation can treat and/or prevent one or more symptoms or effects of the condition of the brain, such as seizures for epilepsy. Furthermore, the neuromodulation can keep the patient in a pre-identified desirable brain state with a high latent space distance from the subclinical onset of undesirable disease symptoms.

At 502, conduction data for a time can be received from at least one recording electrode (e.g., positioned in, on, and/or above the portion of the brain of the patient). The at least one recording electrode can be any number N greater than one and can be sent over N channels to the processor. The at least one recording electrode can be in communication (wired and/or wireless) with the processor (in some instances, in communication with the non-transitory memory which is in communication with the processor).

At 504, the conduction data at the time can be projected through a trained patient-specific model (e.g., trained as shown in FIG. 3) to determine a brain state at the time. The conduction data at the time can be transformed into brain state data at the time that is compatible with the trained patient-specific model (as shown at least in FIG. 4). The transformation can include filtering the conduction data into multi-dimensional time series data signals for the N channels. The filtering can, for instance, include a zero-phase shift 5^{th} order Butterworth filter with passbands of 1 Hz-59 Hz, 61 Hz-119 Hz, and 121 Hz-179 Hz. The conduction data may also be re-sampled to the lowest sample rate of all the conduction data (e.g., if the channels have different sampling rates). For example, if the sampling rate of the conduction data ranges from 512 Hz to 2048 Hz, then the conduction data can all be resampled at 512 Hz to form a multi-dimensional time series data signal.

The trained patient-specific model can then equalize the multi-dimensional time series data signal using a zero-centered one-dimensional histogram equalization (ZHE) scheme to form equalized time series data. For instance, the signal can be split into the positive and negative domains, then a 10,000-bin histogram can be filled for each domain from 0 to the absolute value of highest voltage. Then the linear transfer function from the time series signal to the equalized signal can calculate using the cumulative distribution function for the positive and negative domain separately. The result is a signal that preserves the physiological meaning of zero, but has data evenly distributed between −1 and 1. A normalization scheme can then be applied, such as normalizing all the subject's time series to the first, for instance, 24 hours, of data to produce equalized time series data for each channel.

Then, the equalized time series data can be embedded into a multi-dimensional latent space using an asymmetric recurrent variational autoencoder (AR-βVAE) to form the brain state data in X dimension-latent data space (e.g., having 512 dimensions, but may have any number of dimensions limited only by processing power of the processor and the highest sampling rate of the conduction data) and, in some instances, data forecasting, smooth the latent data (e.g., with a 10-second averaging window and 1 second stride, but any sized-averaging window and/or stride can be used limited by the processing power of the computer). It should be noted that 512 dimensions is far beyond the range of human comprehension.

For instance, the equalized time series data can be compressed into short data epochs (e.g., 0.5 seconds, but can be any short time segment) in 512-dimensional latent space. From there the immediate future (e.g., 0.125 seconds or the like) of all channel data can be simultaneously forecast. The AR-βVAE can be architected to accept N channels of X number of samples (for instance 88-186 channels by 256 samples) and can have multiple layers. The top layer can utilize a Gated Recurrent Unit (GRU) to interface with the input data (the equalized time series data in epochs for the N channels and X samples). The GRU can learn short- and long-range signal features. The GRU can be three layers and bidirectional, with a resulting hidden dimension of 2N that can be subsampled every eight brain states. While not wishing to be bound by theory, this can help the GRU to maintain learning capacity and not be forced to forget data motifs across the forward and reversed sequence lengths. The subsampled hidden states can then be flattened and fed into the β-VAE. The β-VAE can include fully-connected layer feeding into mean and log-variance layers that can be followed by a standard noise-injection reparameterization trick to determine the latent space. The latent space can be regularized by Kulback-Leibler (KL) Divergence and set to a number Y (e.g., 512). The decoder can output an N×64 sample forecast on all channels simultaneously that are compressed into 1×Y (e.g., 512) latent dimensions, which, not wishing to be bound by theory, best embed the necessary information to forecast the next N×64 timepoints in the original input signal. A dropout ratio (e.g., 0.1, 0.2, 0.3, or the like) can be used on the middle fully connected layer of the decoder (e.g., to promote concise and meaningful latent embeddings).

The decoding portion of the β-VAE can be asymmetric, to account for this a custom loss function (called Circular Minimum Hyperbolic Cosine Loss (cMin-LogCosh)) can be applied that allows for varying time shift of the forecasted signals. For instance, the custom loss function can be based on the hyperbolic cosine loss function and the N×64 predicted values can be wrapped in a circle with a stride of one sample and the LogCosh loss calculated every stride resulting in 64 individual loss values. The minimum LogCosh loss can be determined and returned for backpropagation through the model. Additional data transformation may also be used to increase stability, such as learning rate annealing. The 51-dimensional data can also be smoothed with a 10 second averaging window and a 1 second stride to greatly increased the signal-to-noise ratio prior to the manifold approximation.

The dimensionality of the smooth latent data can then be reduced through a pairwise controlled manifold approximation and projection (PaCMAP) (e.g., from 512 to 10), cluster the PaCMAP reduced dimensional space data (e.g., 10 dimensional data, still beyond the range of human comprehension). The reduced dimensional space data can then be fed into a Hierarchical Density Based Spatial Clustering of Applications of Noise (HDBSCAN) for clustering. Distinct brain state grouping(s) for the conduction data at the time can be identified in the reduced dimension and clustered data. The distinct brain state groupings for the time can be projected through the trained patient-specific model. The projection can determine the propensity of the brain state at that time to cause an effect of the condition of the brain. The trained patient specific model does more than compare the current data to previous data, instead the trained patient specific model can analyze the current data with respect to previous data and can even assign a propensity to brain states that have not been seen before based on the analysis. It should be noted that the trained patient-specific model is trained over brain state data from a previous time period of at least one hour, but more preferably at least one day.

At 506, at least one parameter of an electrical signal can be updated based on a propensity of the brain state at the time to cause an effect on the condition of the brain. For instance, if the propensity is considered a high propensity to cause an effect on the condition of the brain (e.g., to cause a seizure or other symptom in the future) then the electrical signal can be modulated (or started if no stimulation was being applied before) to at least partially prevent the effect (or symptom) The processor is in communication with at least a generator that generates the electrical signal and provides the electrical signal to at least one stimulation electrode that applies the electrical signal to the at least another portion of the brain. At 508, the trained patient-specific model can be updated to include the brain state at the time and an effect of the application of the updated at least one parameter of the electrical signal on the brain state at the time. In such a manner the trained patient-specific model is continually updating, adapting, and improving treatment for the patient.

### V. Experiment 1

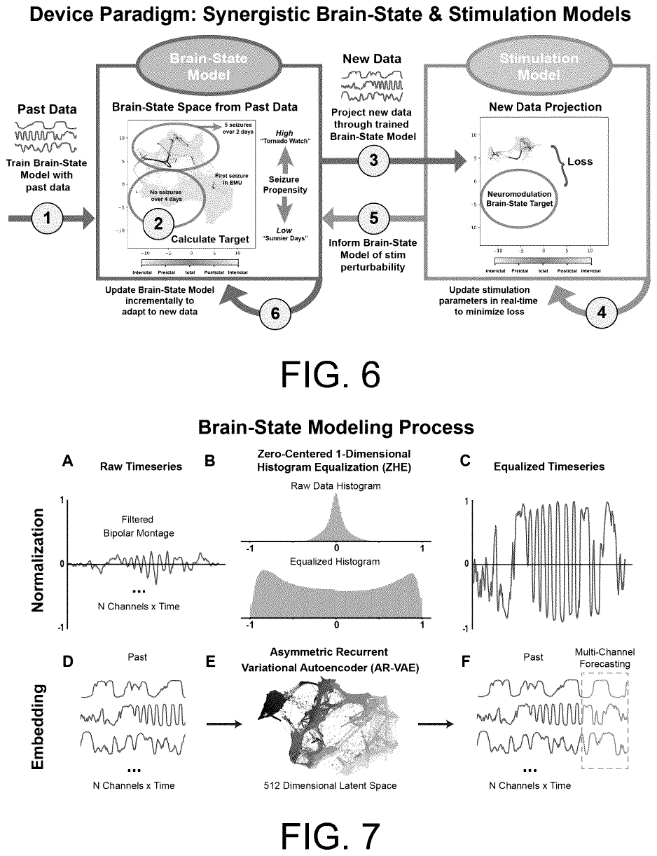

The future of closed-loop adaptive neuromodulation for drug-resistant epilepsy (DRE), as well as other neurological pathologies, relies on effectively quantifying long timescale disease propensity in a smooth and continuous distribution with a functional brain-state for effective device feedback (see example of FIG. 6). The following experiment shows the demonstration and validation of quantifying long timescale disease propensity in a smooth and continuous distribution using stereotactic-electroencephalography (SEEG, a subset of intracranial electroencephalography (iEEG)) timeseries from patients undergoing pre-surgical workup for DRE.

Table 1-contains information about the Epilepsy Brain-State Postulates referenced in this Experimental section.

### Epilepsy Brain-State Postulates

### Definitions

Latent space: The regularized multidimensional space of possible brain-states constructed from previously observed phenomena (e.g. intracranial electroencephalography).
Brain-state/Embedding: The exact mapped location within the latent space. Brain-state is synonymous with ‘embedding’ in this context.
Latent distance: The mathematical distance between two brain-states in the high-dimensional latent space, as defined by metrics like Euclidean, Manhattan, or angular distance. The higher the latent distance, the more ‘different’ the brain states are. Words like ‘far’ and ‘furthest’ are used to indicate brain-states separated by a large latent distance.

### Postulates

### Postulate 1: No Two Pre-Ictal Brain-States are Identical.

Implication: Exact latent space embedding of previous events only serves as approximate landmark for future events. Furthermore, different types of ictal events (e.g. subclinical, focal aware, focal impaired-aware, focal to bilateral tonic-clonic) likely embed into different latent regions.
   Postulate 2: Temporal Evolution from Pre-Ictal to Ictal Brain-State can Vary.
Implication: The exact time course of brain-states in latent space before a given seizure cannot be reliably used as a label for latent space construction or post-hoc interpretation. Specifically, not all brain-states adjacent to a previously verified pre-ictal state will evolve into a seizure in the same manner—there is an inherent level of stochasticity that is difficult to model. Thus, latent space construction benefits from a purely unsupervised/self-supervised model to avoid bias.
   Postulate 3: The Brain-State ‘Furthest’ from Observed Pre-Ictal and Ictal Activity is Ill-Defined.
Implication: In a patient currently experiencing multiple seizures, it is likely that few to no ‘stable’ brain-states far from ictal activity are observed and mapped into the latent space. Thus, a theoretical optimal brain-state target for closed-loop neuromodulation is likely not readily defined by previously observable brain states. Furthermore, there is likely a constellation of potentially stable brain states far from potential ictal activity.

### Methods

The goal of this work was to develop a generalizable framework for a functional brain-state map from high-quality intracranial electrophysiological timeseries to be used with closed-loop neuromodulation. To develop and validate the brain-state model architecture, a cohort of approximately 17,000 hours (16.3 TB of 32-bit precision) of continuous Stereoelectroencephalography (SEEG) data from 118 patients with drug resistant epilepsy (DRE) undergoing SEEG presurgical evaluation at Vanderbilt University Medical Center (VUMC) were utilized. This study was approved by Vanderbilt's Institutional Review Board and all patients provided informed consent.

### Brain-State Modeling.

### Data Acquisition.

SEEG data was collected using Natus Neuroworks (Middleton, WI, USA) with a sampling rate of 512-2048 Hz. All data was resampled down to 512 Hz then filtered using MATLAB's (MathWorks inc., Natick, MA, USA) ‘filtfilt’ function to implement a zero-phase shift 5^{th} order Butterworth filter with passbands of 1 Hz-59 Hz, 61 Hz-119 Hz, and 121 Hz-179 Hz. An adjacent bipolar montage was applied across all SEEG leads and data was saved as 32-bit floating point precision Python pickle files (FIG. 7, element A).

### Zero-Centered One-Dimensional Histogram Equalization (ZHE).

Intracranial electrophysiological timeseries are often low amplitude with rare high amplitude signatures. This can make it difficult for a model to learn nuanced long-range characteristics because the histogram of the data distribution can have long tails (FIG. 7, element B, top panel) representing rare, but physiologically meaningful high voltage signatures. To provide more evenly distributed data suitable for machine learning models, a custom Zero-centered one-dimensional Histogram Equalization (ZHE) scheme was implemented (FIG. 7, element B). This scheme was motivated by traditional histogram equalization for images prior to model training. First, the signal is split into the positive and negative domains, then a 10,000-bin histogram is filled for each domain from 0 to the absolute value of highest voltage. Then the linear transfer function from the raw signal to the equalized signal is calculated using the cumulative distribution function for the positive and negative domain separately. The result is a signal that preserves the physiological meaning of zero, but has data evenly distributed between −1 and 1 (FIG. 7, element C). A prospective normalization scheme must be applied to not violate causality while validating the model on subsequent data epochs, thus all subject's timeseries were normalized to the first 24 hours of data. The histograms for subsequent data epochs will, as expected, not be exactly uniform (e.g. FIG. 7, element B, bottom panel).

### Brain-State Embedding Model.

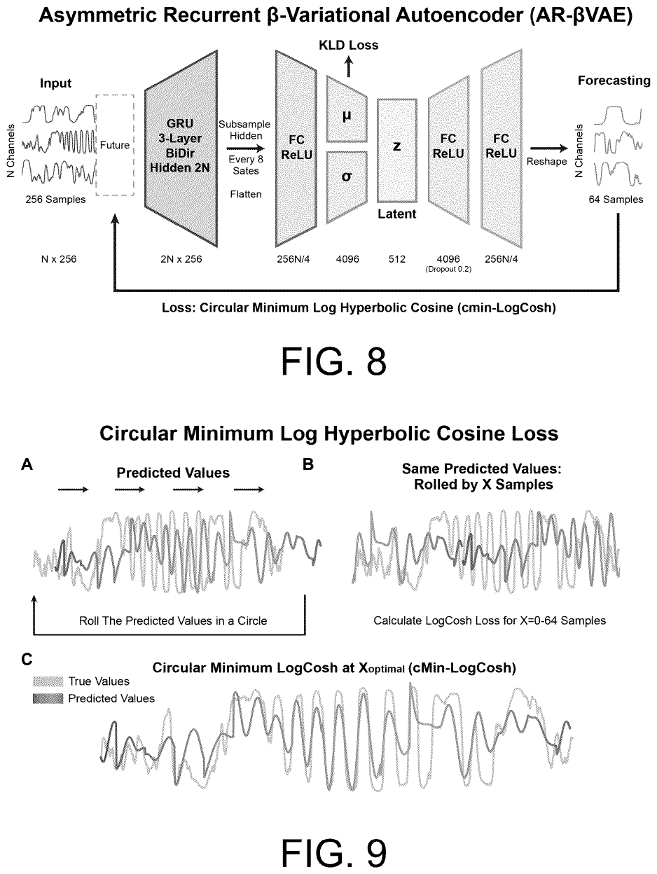

The ZHE data is then used to elucidate brain-states by compressing short (0.5 seconds) data epochs (FIG. 7, element D) into a 512-dimensional latent space (FIG. 7, element E) that is then used to forecast the immediate future (0.125 seconds) of all channel data simultaneously (FIG. 7, element F). This embedding is conducted using a custom Asymmetric Recurrent B-Variational Autoencoder (AR-βVAE), implemented in PyTorch, and trained on the first 70% of a subject's data (FIG. 8). A model was trained for each subject separately to accommodate unique SEEG implants and presumed unique neurophysiological brain-states experienced by each individual.

The AR-βVAE was architected to accept an N-channel (for this dataset, N ranged from 88-168 channels) by 256 sample (i.e. at 512 Hz sampling rate, 0.5 seconds of multichannel data). The top layer of the model utilizes a Gated Recurrent Unit (GRU) to interface directly with the input data and learn short- and long-range signal features. The GRU is three layers and bidirectional, with a resulting hidden dimension of 2N. The hidden state is then subsampled every eight states. In theory, this helps the GRU to maintain learning capacity and not be forced to forget data motifs across the entire 256 forward and 256 reversed sequence lengths. The subsampled hidden states are then flattened and fed into a β-VAE consisting of a fully-connected layer feeding into mean (μ) and log-variance (σ) layers followed by the standard noise-injection reparameterization trick to get ‘z’, the latent space. The latent space was regularized during training with Kullback-Leibler (KL) Divergence. The latent space of the variational autoencoder is set to 512 dimensions. The decoder outputs an N×64 sample forecast on all channels simultaneously. Thus, all N×256 input timepoints are compressed into 1×512 latent dimensions that best embed the necessary information to forecast the next N×64 timepoints in the original input signal. A dropout ratio of 0.2 was used on the middle fully connected layer of the decoder to promote concise and meaningful latent embeddings.

### Circular Minimum Log Hyperbolic Cosine Loss.

Of significance, the decoding portion of the β-VAE is asymmetric because the input GRU layer in the encoder cannot be easily reversed for the decoder. This creates an undesirable situation where slightly shifted input signals could require dramatically different embeddings due to the rigidity of the output fully-connected decoding layers—e.g., small time shifts in the input data could require very different outputs from the final fully connected layers. To accommodate this asymmetric intractability, a custom loss function (FIG. 9) was developed that allows for a varying time shift of the forecasted signals, termed Circular Minimum Hyperbolic Cosine Loss (cMin-LogCosh). This loss function was based on the hyperbolic cosine loss function as implemented in PyTorch by ‘auraloss’ package. Specifically, the N×64 predicted values are wrapped in a circle using ‘torch.roll’ in PyTorch with a stride of one sample (FIG. 9, elements A and B) and the LogCosh loss calculated every stride resulting in 64 individual loss values. The minimum LogCosh loss is deemed the cMin-LogCosh (FIG. 9, element C) and returned for backpropagation through the model.

### Training Considerations & Kullback-Leibler/Learning Rate Annealing.

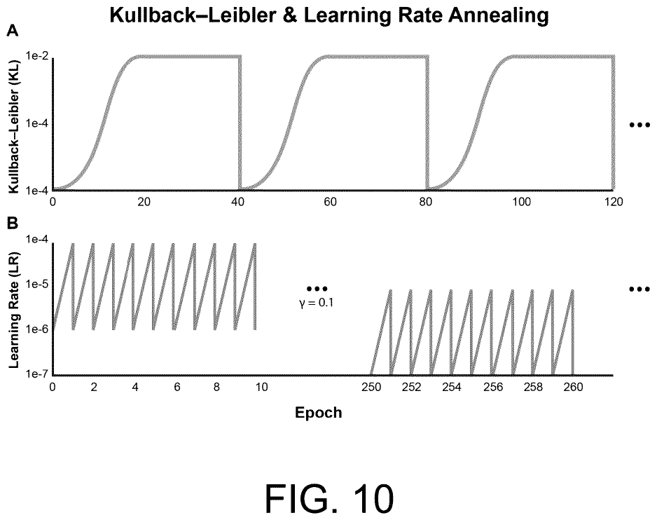

Each subject's model was trained by feeding in random N×256 epochs of data with a batch size of 64. A total of 640,000 random epochs and the models were each run to a total of 500 epochs, resulting in a total of 320M random epochs used for training. The first 70% of the patient's data was used for training and the remaining 30% was completely left out and used in the validation sections as described below. In an attempt to maximize biologically meaningful features extracted from the dataset, a sigmoidal KL annealing schedule was implemented with a 40 epoch period (FIG. 10, element A). Additionally, to increase model training stability, a learning rate (LR) annealing with a single epoch saw-tooth waveform and a gamma of 0.1 at 250 of the total 500 training epochs was implemented (FIG. 10, element B).

### Brain-State Interpretation: Dimensionality Reduction & Clustering.

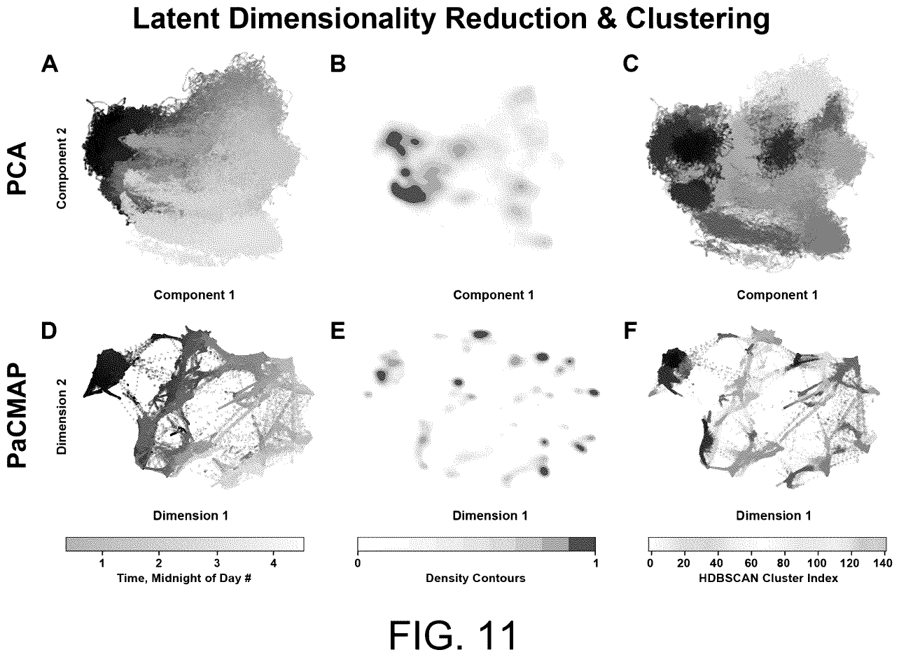

After model training has completed, the entire training and validation datasets (70/30%) are sequentially run through the model to get a continuous 512 dimensional latent-space representation of the data. These high-dimensional timeseries data are impossible for a human to interpret; thus, two steps are required before biologically meaningful analyses can be conducted: 1) Dimensionality reduction, 2) Brain-state clustering. To begin, dimensionality reduction is critical to interpreting the latent space architecture, but traditional methods like Principal Components Analysis (PCA) fail to separate brain-states into any discernible structure (FIG. 11, elements A-B). Thus Pairwise-Controlled Manifold Approximation and Projection (PaCMAP), a dimensionality reduction algorithm and successor to the popular Uniform Manifold Approximation and Projection (UMAP) were used, that has demonstrated enhanced ability to capture global data structure, which is critical for analyzing large-timescale brain states (FIG. 11, element D). Before feeding the latent space data into PaCMAP, the latent space data were smoothed the 512-dimensional latent data with a 10-second averaging window, and 1 second stride—it was found that this greatly increases the signal-to-noise ratio for the manifold approximation. The PaCMAP hyperparameters that differed from default were as follows: Distance: cosine, MN_ratio 2.0, FP_ratio 0.5 to enhance global structure definition of the reduced space.

Next, differing densities of data appeared to be present in the data, as can be appreciated in the iso-density contours in FIG. 11, elements B and E. Thus, the PaCMAP reduced dimensional space was clustered using Hierarchical Density-Based Spatial Clustering of Applications with Noise (HDBSCAN). Of note, two separate runs of PaCMAP were conducted, the first is the 512 dimensional latent space reduced to a ten-dimensional space that was fed into HDBSCAN, and the second is a two-dimensional space for direct visualization in the plots shown. This was done to allow HDBSCAN to have access to an enriched ten-dimensional feature set that could help distinguished unique brain states without being limited to the simplified two-dimensional space. The hyperparameters that differed from default for HDBSCAN were as follows: min_cluster_size 200 (each data point is a 10 second average of latent data), min_samples 100. After dimensionality reduction and clustering, distinct brain-state groupings within the latent space become apparent in the 2D PaCMAP visualization (FIG. 11, element F), that would have otherwise been indistinguishable by PCA (FIG. 11, element C). Of note, the PaCMAP and HDBSCAN models were built with only training data.

Next, using the trained AR-βVAE, PaCMAP and HDBSCAN models, the completely withheld validation data can be run through the pipeline to observe the most-likely brain-state cluster for data the models have never previously seen. This flow of data through the pretrained models is how a real-time feedback signal would be generated for a closed-loop neuromodulation device. An ‘adaptive’ paradigm would arise from continuously updating these models based on new data to refine the brain-state estimation in real time.

### Brain-State Model Validation: Perturbability & Biological Relevance.

The generation of clustered latent spaces from epiphenomenal physiological timeseries has little meaning to potential closed-loop neuromodulation applications without two important forms of validation: 1) Evidence of brain-state perturbability with neurostimulation, 2) Evidence of biological relevance of brain-states. Both of these validation conditions must be reasonably met to proceed with a closed-loop neuromodulation paradigm. However, the exact criteria that dictates adequate validation is nebulous-especially establishing biological relevance because this is subject and disease specific. For example, the latent space of a person with epilepsy may differ greatly from a person with major depressive disorder. Or more nuanced, the difference between two people with epilepsy who experience different seizure types, take different medications, and are different ages. Thus, the following quantitative and qualitative techniques were implemented to capture the subject-to-subject variability in brain-state organization.

Brain-State Perturbability with Low-Frequency Neurostimulation.

To capture possible effects of neurostimulation, previously collected single-pulse electrical stimulation (SPES) paradigm conducted with 71 of 118 pre-surgical epilepsy patients were utilized. This stimulation paradigm was not designed to maximize any possible brain-state modulation and consists of only 1 Hz, 300 microsecond biphasic pulses in trains of 10 seconds and at current levels of 1-5 milliamps. Thus, these SPES sessions offer a conservative insight into the effects of a low-energy stimulation paradigm's effects on brain-state. The SPES data epoch (approximately 1-2 hours per subject) was excluded from training. Inference of the SPES epoch was conducted without manual stimulation artifact removal to minimize potential bias and simulate a real time device implementation-artifact is greatly mitigated by the ZHE normalization scheme and with the brevity of SPES artifact accounting for approximately 0.1%-0.2% of any smoothed 10 second window. For continuous stimulation paradigms with a higher duty cycle, this technique may need to be amended.

To quantify any effects of neurostimulation, two high-level metrics: 1) number of brain-state transitions within a 60 second window, 2) number of unique brain-states occupied within a 60 second window were implemented. These metrics serve as a baseline validation to capture coarse information about the effects of neurostimulation. Importantly, due to the architecture of each model in a subject-specific manner, it is ill-posed to compare these metrics across subjects. Thus, a bootstrapping technique was implemented to estimate the statistical significance for each subject. Specifically, the ‘state-transitions’ and ‘unique-clusters’ metrics were evaluated for all non-overlapping 60-second windows within the SPES epoch, then calculated the metrics for an equivalent number of random 60-second epochs in the pre-SPES epoch. From these bootstrapped runs, 95% confidence intervals and two-sample t-tests can be conducted with Bonferroni-Holm multiple comparison correction.

### Biological Relevance of Brain-States.

Thus far, all methodology has been left agnostic to any specific neurological disease. However, for the purpose of determining the biological relevance of the elucidated brain-states, the latent spaces must be interpreted in the specific context of the cohort's neurophysiological pathology-drug-resistant epilepsy. This type of validation relies on qualification of the latent space structure with detailed clinical information known about each subject. Most importantly for this cohort, the exact timing and type of all electroclinical seizures. With these events as ground truth, it can be worked backwards to overlay meaning to the brain-states that precede and follow these events.

With the epilepsy brain-state postulates in mind from Table 1, three qualitative questions were asked on the training data and validation data withheld from each subject's trained model: 1) Does pre-ictal, ictal, and post-ictal activity in training data aggregate together in the latent space in a meaningful structure, and are any patterns based on different seizure types present? 2) Does completely withheld validation data for similar electroclinical epileptic events map to the expected latent space regions based on training data latent space assessment? 3) Can seizure propensity be assigned to the HDBSCAN clusters and plotted on a timeline for training data which can then be mapped to validation data?

To address the first and second validation questions, the entire training and validation datasets were run through the trained AR-βVAE model and the latent spaces for each subject were labeled with known ictal events. The latent space density iso-contours were concurrently plotted to assess the most common brain-states during the training and validation epochs. Next, the latent architectures during ictal and interictal periods were analyzed for meaningful aggregation of known electroclinical states. Finally, to assess the seizure propensity of the latent space clusters (i.e. brain-states), a timeline of cluster assignments for the training dataset was plotted against known ictal events. An exponentially decaying seizure propensity scoring mask (see Eq. 1, where ‘t’=sample count to seizure at sampling rate of 512 Hz, and d=decay factor of 20,000) was assigned to the timeline for all ictal events, with the maximum mask value at each timepoint was used to account for temporally adjacent ictal events.

$$Seizure Propensity Scoring Mask = {e}^{( \frac{- t}{d} )} \qquad Eq 1.$$

The brain-state clusters were then ranked by the mean scoring mask value of all time periods during which the cluster was assigned. With the cluster seizure propensity score assigned from the training dataset, the validation scored timeline can be plotted to assess the ability of the model to discern potentially high seizure risk brain-states on data the model has never seen before.

### Results

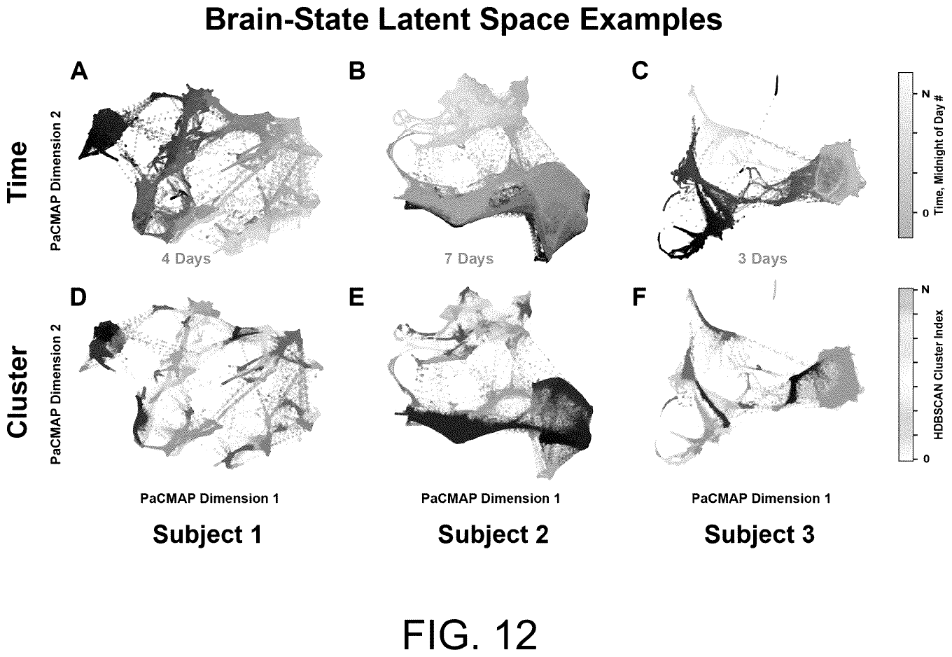

The main goal of this experiment is to provide an initial validation of the methods of brain-state modeling using high-level quantitative and qualitative metrics to assess proof-of-concept suitability for an adaptive closed-loop neuromodulation paradigm. Utilizing this modeling process, example latent spaces and brain-state clusters can be seen in FIG. 12.

### Brain-State Observed to be Significantly Perturbed by Low-Frequency Stimulation.

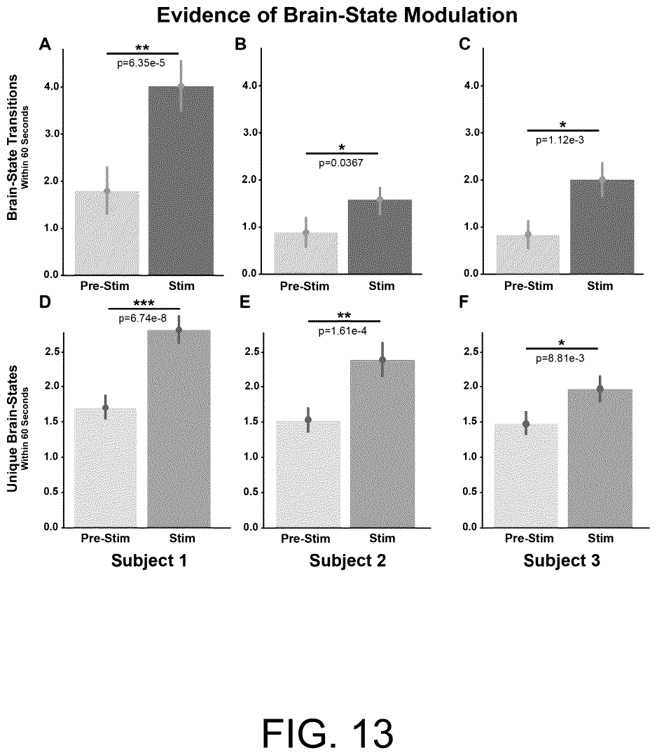

The results of the neurostimulation perturbation analysis for these example subjects can be seen in FIG. 13. SPES displayed evidence of significant brain-state perturbation, with the mean number of brain-state transitions observed to be 177%-250% higher during the SPES epoch: Bootstrapping t-test p-values ranged from 6.35e-5 to 0.0367 for the average number of brain-state transitions during a given 60 second window in the pre-SPES vs. SPES epochs. Furthermore, the increase in brain-state transitions during the SPES epochs were not simply observed to be oscillations between the same brain-states because the number of unique brain-states increased by 127%-163% during the SPES epochs, with p-values ranging from 6.74e-8 to 8.81e-3. These results indicate that even though SPES is a low-energy stimulation paradigm, it may still be significantly altering the brain-state as captured by the proposed brain-state modeling process.

### Brain-States Appear to be Biologically Relevant Based on Inspection of Latent Space Architecture.

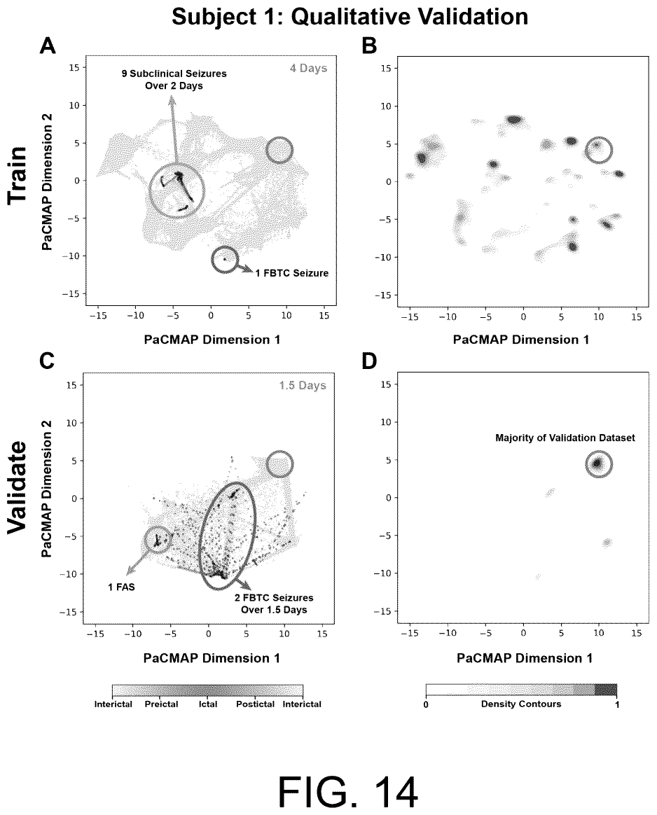

Beyond the ability to capture possible effects of neuromodulation, the brain-state modeling process must capture biologically meaningful information for a given neuropathology to be of clinical utility. To assess biological relevance, the qualitative assessment of the latent spaces for three diverse subjects with epilepsy was outlined (FIGS. 14-16). Two subjects experienced focal to bilateral tonic-clonic (FBTC) seizures during SEEG recording (Subjects 1-2) and the third (Subject 3) experienced only subclinical and focal aware seizures (FAS). Of further interest, Subjects 1-2 had multiple seizure-free days prior to the first seizure captured on SEEG recording, whereas Subject 3 had multiple subclinical and FAS immediately on the first day of recording. These are important factors to consider when looking at the latent spaces for these subjects.

To begin, Subject 1's latent space for the training data (FIG. 14, elements A-B) displays a distinct grouping of nine subclinical seizures that occurred over a two-day period. Furthermore, the sole FBTC seizure present in the training dataset is well separated from this subclinical aggregation, providing evidence that the pre-ictal brain-state prodrome for the FBTC seizure differs to that of the subclinical seizures. However, these observations are for the data that the model had seen during training, thus analysis of the validation data completely withheld from the model training is of more interest. The validation latent spaces for Subject 1 (FIG. 14, element C) at first appear noisy, but when looking at the density iso-contours of brain-states (FIG. 14, element D), the majority of the validation brain-states were distant to areas of the latent space that were labeled as ictal in the training latent space. Furthermore, the two FBTC seizures present in the validation data mapped well to the training FBTC latent space despite only a single FBTC seizure being present for training. Finally, a new seizure type was present in the validation data (FAS) that mapped to a distinct location in the latent space near the subclinical aggregation despite this seizure type never having been seen by the model for training. This subject serves as a nice example of the potential variety in pre-ictal brain-states for different types of seizures, as outlined in the implications of Postulate 1.

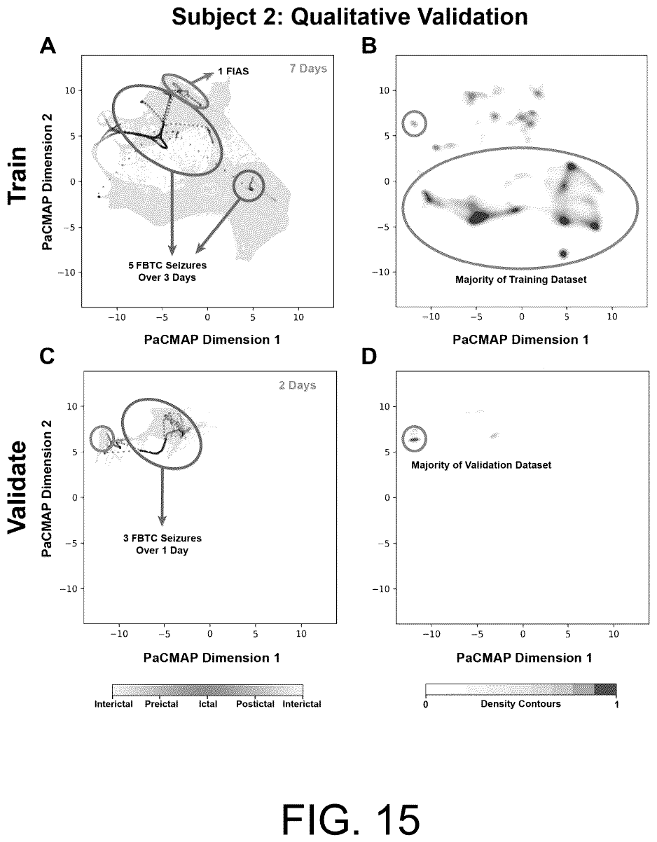

The next example subject also had many seizure-free days prior to the first electroclinical event, however this patient's training data had five FBTC seizures present in the training data (i.e., first 70% of data), thus this subject's model was exposed to many more brain-state evolutions toward a FBTC seizure (FIG. 15, elements A-B). This is likely reflected in the tight aggregation of FBTC seizures in the validation data (FIG. 15, element C) to the expected highest density of FBTC seizures in the training data (FIG. 15, element A). Notably, the FTBC seizure in the training latent space that is most distant from the other four FBTC seizures was the first electroclinical event that the patient experienced during recording-perhaps triggering the subject to enter the high seizure propensity brain-states that resulted in four FBTC seizures over the next 2 days. An observation that provides further evidence for the subject's overall brain-state shift throughout recording is the very different latent space occupancy during training (FIG. 15, element B) compared to validation (FIG. 15, element D). This serves as a good example of potentially different pre-ictal brain-state evolutions that can occur, as outlined in Postulate 2. Finally, this patient also experienced a focal impaired-aware seizure (FIAS) that mapped adjacent to the FBTC seizures in the training latent space, but no FIAS were present in the validation data to assess generalizability.

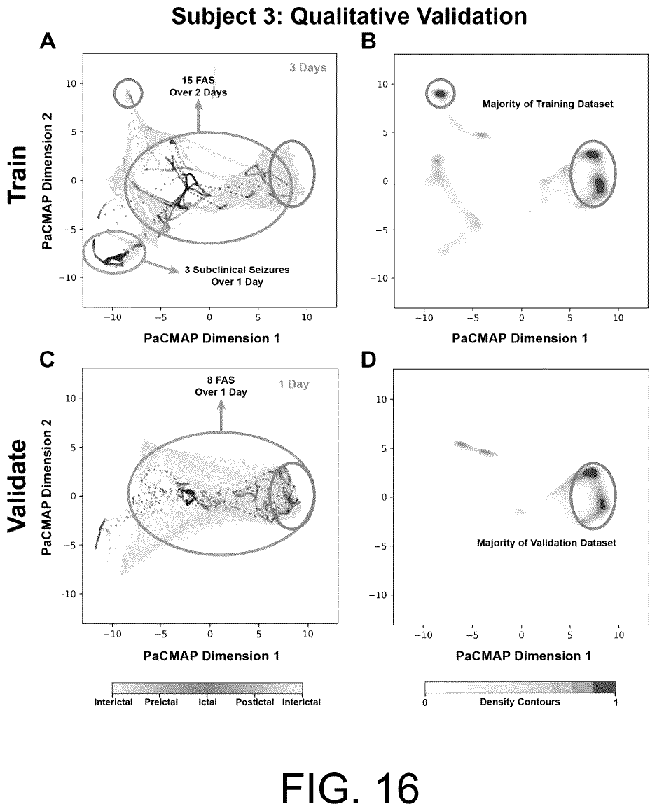

The last example, Subject 3, had a significantly different electroclinical timeline of ictal events during SEEG recording, which is reflected in the training and validation latent spaces (FIG. 16). The first observation is that the iso-density contours for training (FIG. 16, element B) and validation (FIG. 16, element D) are similar—this indicates that unlike Subjects 2 & 3, this subject likely did not experience a significant shift in the constellation of brain-states during recording. Thus, this subject serves as an exemplary case for the implications of Postulate 3 that there may be no stable inter-ictal brain-state mapped into the latent space if the subject is experiencing a high ictal burden during recording. As suspected, unlike Subjects 1 & 2 with a more concentrated aggregation of ictal activity in the latent space, Subject 3's latent space is dominated by numerous FAS and subclinical seizures (FIG. 16, element A). Importantly, the peri-ictal activity for the three subclinical and 15 FAS in the training data did map to different areas of the latent space, but the FAS tend to dominate the majority of the space. Evidence for this patient-specific hypothesis is observed in the validation latent space (FIG. 16, element D), where the eight FAS present in the validation data occupy a similar, but also expansive, area of the latent space similar to the training data. For the purposes of brain-state modeling, this patient would have benefitted from a longer recording time to hopefully capture periods of lower seizure burden. Thus, as Postulate 3 states, it may be difficult to extrapolate a ‘stable’ interictal brain-state that is far in latent space from ictal activity if no stable state has been previously observed.

Seizure propensity timeline demonstrates large-timescale transition to high ictal burden.

An important feature of a potential closed-loop neuromodulation device for drug-resistant epilepsy is the ability to sense large-timescale shifts in brain-state. Without this ability, the device operates in one of two paradigms: 1) The “too little too late” paradigm where only an immediate pre-ictal signature can be detected and stim can be delivered, or 2) The device inappropriately characterizes large-timescale brain-states and thus operates in essentially an open-loop fashion with inadequate feedback signaling.

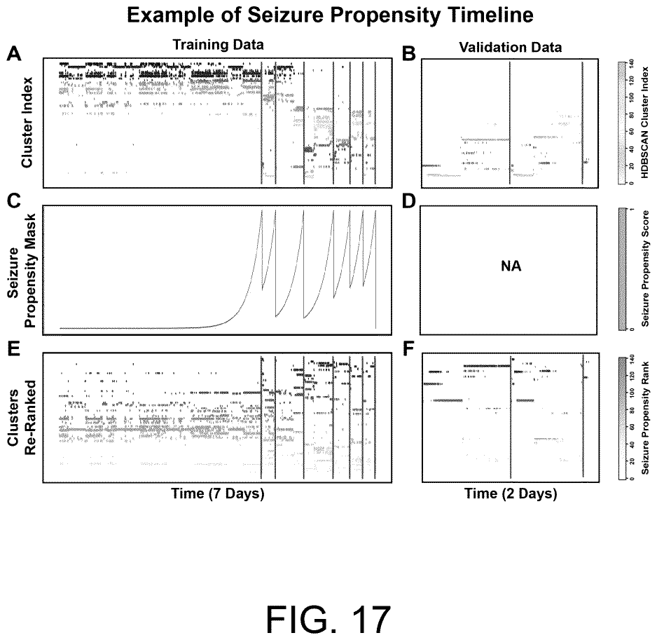

An example of the proposed brain-state modeling process ability to capture large-timescale brain-states is shown in FIG. 17 where example Subject 2's seizure propensity timeline is plotted for training and validation data. This subject serves as a good example because the training data consists of many days of seizure-free recordings preceding multiple days of frequent FBTC seizures (FIG. 17, element A), which were continued in the validation data (FIG. 17, element B). Thus, using the seizure propensity masking technique (FIG. 17, element C), the brain-state clusters can be ranked to align with estimated seizure-propensity within an arbitrary timeframe (FIG. 12, element E), high values indicate high risk for a seizure. The validation timeline for this subject appropriately suggests that this patient spent most of the two days of validation recordings in a high seizure propensity state-which is in alignment with the two FBTC seizures this patient experience during validation. It is important to note that these are not immediately pre-ictal states and could be operated on by a neuromodulation device on the order of hours-days in an attempt to neuromodulate this subject through their mapped latent space to previously detected clusters with a lower seizure propensity.

### Invention 2—A Generalized Model and Use Thereof on Previously Unseen Patients

### VI. Systems 2

It should be understood that Systems 2 (e.g., system 150) can include the same physical components as described with respect to Systems 1 (e.g., system 100). Accordingly, the system 150 can be described with reference to FIGS. 1 and 2.

As shown in FIG. 1, the system 150 can include one or more recording electrodes (recording electrode(s) 102) (e.g., a plurality of recording electrodes) that can record conduction information from at least a portion of a patient's brain (shown in FIG. 1 as the dashed box labeled brain). It should be understood that in reference to system 150 (e.g., System 2) the patient can be a previously unseen patient (e.g., a patient who has not been previously seen by the brain state model). Conduction data can refer to electrical activity recorded from the patient's brain. For example, the conduction data can be recorded using electroencephalography (also referred to as EEG), a test that measures electrical activity a patient's brain. Recording electrode(s) 102 can be at least one of external, internal, intracranial, or the like. For instance, the recording electrode(s) 102 can include one or more intracranial electrodes for intracranial EEG (also referred to as iEEG) or stereoelectroencephalography (also referred to as SEEG). In another instance, the recording electrode(s) 102 can be one or more electrodes implanted over the cortex in subdural space for electrocorticography (ECOG). In a further instance the recording electrode(s) 102 can be scalp electrodes configured for scalp EEG recording. In each case, the conduction data can provide a representation of electrical activity in one or more areas of the brain of the patient at a given time. The recording electrode(s) 102 can be any number N that is one or greater (e.g., a plurality).

Conduction data recorded by the recording electrode(s) 102 (N channels of data corresponding to the N recording electrodes 102) can be sent to controller 106 (e.g., that stores and executes the generalized brain state model in system 150). The conduction data can be recorded and transmitted at one or more frequencies. The recording electrode(s) 102 and the controller 106 can be in wired and/or wireless electrical communication. The controller 106 can include memory 108 (which is non-transitory) and a processor 110 (e.g., a microprocessor, a computing device, a state machine, a signal processing chip, or the like). In some instances, at least a portion of the memory 108 and processor 110 can be embedded within the same device (e.g., a microprocessor). In other instances, the memory 108 and the processor 110 can be entirely separate devices. In some instances the controller 106 can be one or more controllers each with a memory 108 and a processor 110 that can store and/or execute at least a portion of the instructions and/or the brain state model. The memory 108 can store a trained brain state model and instructions related to executing the trained brain state model. The processor 110 can execute the instructions and the generalized trained brain state model, for instance, to determine brain states from the conduction data, whether to apply electrical neuromodulation (e.g., via the stimulating electrode(s)/device(s) 114), and if electrical neuromodulation is applied which one or more parameters of the neuromodulation to alter to update the neuromodulation treating the condition of the brain (more detail shown in FIG. 2). The controller 106 may also, in some instances, have or be in communication with (wired and/or wireless) a display and/or output device 112 (or other type of output device for visual, audible, or tactile output) and/or an input device (e.g., use interface) (not shown, for inputting one or more instructions, limits, additional data, or the like).

Upon determining that at least a portion of the conduction data at a time corresponds to a brain state indicating a condition, a need for neuromodulation, and/or a change in neuromodulation (e.g., a high propensity brain state, described in further detail below), the controller 106 can determine (if no neuromodulation is being applied) or update (if neuromodulation is already being applied) one or more parameters of an electrical signal (e.g., corresponding to shape, current, voltage, amplitude, frequency, pulsation, timing, etc.) and send the one or more new or updated parameters to a generator 104. The generator 104 may be a standalone device in electrical communication (wired and/or wireless) with the controller 106 and the stimulating electrode(s) and/or stimulating device(s) 114 (as shown), part of the controller 106, part of a system including the stimulating electrode(s) 114, etc.). The generator 104 can receive the one or more new or updated parameters and create or update the electrical signal based on the new or updated parameters. The electrical signal can be sent to one or more stimulating electrodes (stimulating electrode(s)/stimulating device(s) 114) for application of the configured electrical signal to at least another portion of the brain for neuromodulation. The at least the other portion of the brain can include at least a portion of the same portion of the brain monitored by the recording electrode(s) 102, different from the portion of the brain monitored by the recording electrode(s), and/or at least partially the same and at least partially different. The stimulating electrode(s) 114 can be internal electrodes (e.g., intracranial electrodes) and/or surface electrodes (e.g., positioned on the scalp). In some instances, the recording electrode(s) 102 and the stimulating electrode(s) 114 can be the same electrodes. In other instances, the recording electrode(s) 102 and the stimulating electrode(s) 114 can be unique and distinct from one another. While not shown, it should be understood that the controller 106 may also be in communication with one or more other types of treatment devices, e.g., pharmaceutical delivery devices, other stimuli delivery devices (e.g., heat, cold, light, etc.), or the like, in place of the generator 104 and stimulating electrode(s)/device(s) 114) that can provide treatment that can stimulate and/or suppress/inhibit neural conduction.

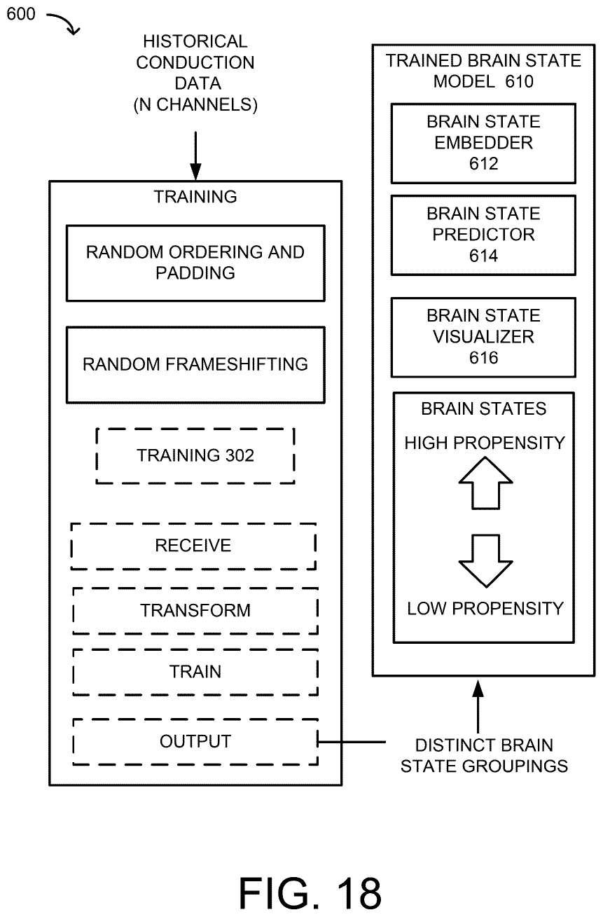

In some instances, the system 150 can be a closed-loop between the recording electrode(s) 102, the controller 106, and the generator 104/stimulating electrode(s) 114. To implement the neuromodulation in the closed-loop, the controller 106 can implement the trained brain state model to determine a propensity of a brain state at a given time towards one or more effects or symptoms of a condition of the brain of the patient and determine whether that brain state should trigger treatment and/or a change to a current treatment. An example of the functionality of the controller 106 is shown in FIG. 2, further details for system 150 are discussed with respect to FIG. 18. Conduction data recorded by the recording electrodes can be received at a time (receive 202, which may be conducted by a brain state embedder). The conduction data can then be projected through the trained brain state model to determine a brain state at the time and the brain state's propensity (project 204, which may be conducted by a brain state predictor). For example, the processor 110 can execute the trained patient-specific model and can process the conduction data at the time into brain state data at the time to be compatible with the trained brain state model The brain state data can then be projected through the trained brain state model to determine the propensity of the brain state at the time and whether that brain state should trigger treatment and/or a change to a current treatment.

The trained brain state model can employ a multi-dimensional latent space to determine whether the brain state at the time has a propensity to cause an effect on a condition of the brain (e.g., a neurological pathology, such as epilepsy). For instance, the brain state at the time can be run through the multi-dimensional latent space and analyzed with respect to a multitude of brain states with known effects on the condition of the brain to determine the current propensity to cause an effect on the condition of the brain. It should be noted that the this is far from a simple one-to-one comparison (or even a plurality of one-to-one comparisons) but instead includes multi-dimensional comparisons of positions and calculations of a plurality of interacting signs and brain states that a person cannot accomplish in the mind. As an example, the positions can correspond to cautions similar to tornado watches and warnings. In one area, the positions can correspond to no watch (low propensity), another area can correspond to a watch if some early signs appear (mid-level propensity), while another area can correspond to a warning when more or stronger signs appear (high propensity). It should be understood that these are only examples, and that the propensity can be on a gradient that can include many different propensities that may or may not be pointed in nature. The trained brain state model can include historical data with known outcomes for the comparison but need not include each of the specific brain states being compared during use as the patient is a patient previously unseen by the model. Additionally, the trained brain state model is able to predict propensity even for brain state data that it has not yet seen (e.g., from patients that were previously unseen) as it is believed that no two brain states are entirely identical in latent space. It should be further noted that, while not wishing to be bound by theory, the exact time course of brain states in latent space before a given brain event (e.g., a seizure) cannot be reliably labeled (e.g., will or will not result in an effect) so propensity is determined rather than categorization of brain states (e.g., by brain state predictor). When the brain state indicates a propensity to cause an effect on the condition of the brain at least one parameter of the electrical signal for the neuromodulation (parameter(s) 208) and, in some instances, the trained brain state model (model 210) can be updated (updated 206) although the trained brain state model need not be updated. As part of the updating the at least one parameter can be updated and sent to the generator via the processor 110 (e.g., via a brain state visualizer) and the trained brain state model can be updated to include that brain state, the effect on the patient, and/or the effect of the change in the one or more parameters on the patient to become a trained patient specific model.

FIG. 18 shows an example 600 of how the trained brain state model 610 can be trained in order to be used for determining current brain states (and/or propensities of current brain states) and/or predicting future brain states (and/or propensities of future brain states) of a previously unseen patient). The training can occur before the model is used (e.g., using historical data from the patient, historical data from patients suffering from the same general condition, etc.). In some instances, the brain state model 610 can be continued for each “update” during implementation of the model for closed-loop neuromodulation. However, the neuromodulation may also be conducted in an open-loop fashion.

For example, historical conduction data (from N-channels corresponding to N recording electrodes) from other patients from a given previous time period can be input into a training module. The training can, in some instances, include all or some of the steps discussed in FIG. 3 with respect to training module 302). During training the model can receive the historical conduction data and filter the historical conduction data into multi-dimensional time series data signals for the N channels. The filtering can, for instance, include a zero-phase shift 5^{th} order Butterworth filter with passbands of 1 Hz-59 Hz, 61 Hz-119 Hz, and 121 Hz-179 Hz. The historical conduction data may also be re-sampled to the lowest sample rate of all the historical conduction data. For example, if the sampling rate of the historical conduction data ranged from 512 Hz to 2048 Hz, then the historical conduction data can all be resampled at 512 Hz. The training 302 module can then equalize the multi-dimensional time series data signal using a zero-centered one-dimensional histogram equalization (ZHE) scheme to form equalized time series data. For instance, the signal can be split into the positive and negative domains, then a 10,000-bin histogram can be filled for each domain from 0 to the absolute value of highest voltage. Then the linear transfer function from the time series signal to the equalized signal can be calculated using the cumulative distribution function for the positive and negative domain separately. The result is a signal that preserves the physiological meaning of zero, but has data evenly distributed between −1 and 1. A normalization scheme can then be applied, such as normalizing all the subject's time series to the first, for instance, 24 hours, of data to produce equalized time series data for each channel.

Then, the training 302 module can embed the equalized time series data into a multi-dimensional latent space using an asymmetric recurrent variational autoencoder (AR-βVAE) to form the brain state data in X dimension-latent data space (e.g., having 512 dimensions, but may have any number of dimensions limited only by processing power of the computer and the highest sampling rate of the conduction data) and, in some instances, data forecasting, smooth the latent data (e.g., with a 10-second averaging window and 1 second stride, but any sized-averaging window and/or stride can be used limited by the processing power of the computer). It should be noted that 512 dimensions is far beyond the range of human comprehension.

For instance, the equalized time series data can be compressed into short data epochs (e.g., 0.5 seconds, but can be any short time segment) in 512-dimensional latent space. From there the immediate future (e.g., 0.125 seconds or the like) of all channel data can be simultaneously forecast. The AR-βVAE can be architected to accept N channels of X number of samples (for instance 88-186 channels by 256 samples) and can have multiple layers. The top layer can utilize a Gated Recurrent Unit (GRU) to interface with the input data (the equalized time series data in epochs for the N channels and X samples). The GRU can learn short- and long-range signal features. The GRU can be three layers and bidirectional, with a resulting hidden dimension of 2N that can be subsampled every eight brain states. While not wishing to be bound by theory, this can help the GRU to maintain learning capacity and not be forced to forget data motifs across the forward and reversed sequence lengths. The subsampled hidden states can then be flattened and fed into the β-VAE. The β-VAE can include fully-connected layer feeding into mean and log-variance layers that can be followed by a standard noise-injection reparameterization trick to determine the latent space. The latent space can be regularized by Kulback-Leibler (KL) Divergence and set to a number Y (e.g., 512). The decoder can output an N×64 sample forecast on all channels simultaneously that are compressed into 1×Y (e.g., 512) latent dimensions, which, not wishing to be bound by theory, best embed the necessary information to forecast the next N×64 timepoints in the original input signal. A dropout ratio (e.g., 0.1, 0.2, 0.3, or the like) can be used on the middle fully connected layer of the decoder (e.g., to promote concise and meaningful latent embeddings).

The decoding portion of the β-VAE can be asymmetric, to account for this a custom loss function (called Circular Minimum Hyperbolic Cosine Loss (cMin-LogCosh)) can be applied that allows for varying time shift of the forecasted signals. For instance, the custom loss function can be based on the hyperbolic cosine loss function and the N×64 predicted values can be wrapped in a circle with a stride of one sample and the LogCosh loss calculated every stride resulting in 64 individual loss values. The minimum LogCosh loss can be determined and returned for backpropagation through the model. Additional data transformation may also be used to increase stability, such as learning rate annealing. The 512 dimensional data can also be smoothed with a 10 second averaging window and a 1 second stride to greatly increased the signal-to-noise ratio prior to the manifold approximation.

The training 302 module can then reduce the dimensionality of the smooth latent data through a pairwise controlled manifold approximation and projection (PaCMAP) (e.g., from 512 to 10), cluster the PaCMAP reduced dimensional space data (10 dimensional data, still beyond the range of human comprehension). The reduced dimensional space data can then be fed into a Hierarchical Density Based Spatial Clustering of Applications of Noise (HDBSCAN) for clustering. Distinct brain state grouping(s) for the historical data can be identified in the reduced dimension and clustered data. The historical conduction data over which the trained patient-specific model is trained can be brain-state data for a previous time period (e.g., of more than 10 minutes, 30 minutes, 1 hour or more, 1 day or more, 3 days or more, etc.). The distinct brain state groupings discovered by the training 302 module can be used to form the high propensity to low propensity designations (e.g., on a gradient).

Alternatively and/or additionally, the training can include one or more steps different from the training module 302. The training can include, but is not limited to receiving, by a system comprising a processor, raw conduction data of a brain from a plurality of patients; transforming the raw conduction data into a 1024 dimensional vector; training a Gaussian-mixture variational auto-encoder (GM-VAE) with an adversarial estimation of Kullback-Leibler divergence; outputting a clustered, smooth latent space of blended brain states across patients; and training a brain state predictor on embeddings of the clustered, smooth latent space of the blended brain states of the plurality of patients to predict future brain states. Training the brain state model can also include randomly ordering and padding with zeros up to 256 channels for every forward pass of the conduction data from the plurality of patients, by the system, through the trained brain state model, and randomly frameshifting, by the system, current and predicted brain state conduction data of the plurality of (known) patients to provide a more generalizable model.

The trained brain state model 610 can include at least a brain state embedder 612, a brain state predictor 614, and a brain state visualizer 616. The trained brain state model 610 can include a high propensity/low propensity brain state ranker, as well that can indicate the likelihood of the brain state and/or the future predicted brain state being correct. The trained brain state model can be embodied on at least one controller (e.g., 106 and can be stored in memory and executed as instructions be the processor). As noted, the trained brain state model 610, notably, is not trained on the conduction data of the previously unseen patient so is not considered customized for that patient (e.g., saving significant time and resources). As previously described, the trained brain state model 610 can be trained to generalize the conduction data of the previously unseen patient with a heavily randomized training paradigm that includes at least: randomly ordering and padding with zeros up to a number of channels of a maximum number of the plurality of electrodes for every forward pass of known subject data through the trained brain state model, and randomly frameshifting current and predicted brain state conduction data of the known subjects.

A previously unseen patient can provide conduction data to the trained brain state model 610. The brain state embedder 612 can receive an input of the conduction data from the plurality of recording electrodes. The conduction data can, in some instances be pre-processed, including referencing the conduction data received from the plurality of electrodes; filtering the conduction data with infinite impulse response zero-phase digital filters; and optimizing a data distribution of the conduction data. The brain state embedder 610 can embed the conduction data into a common latent space with a number of dimensions (e.g., 1024). As an example, the brain state embedder 612 can form outputs having at least embedded conduction data in the common latent space (the common latent space can be of any size, such as 1024 dimensions). For example, the brain state embedder 612 can reference the conduction data received from the plurality of electrodes (e.g., with an adjacent bipolar montage); filter the conduction data with infinite impulse response zero-phase digital filters (e.g., using pass windows of 1-59, 61-99, 121-179 Hz); and optimize a data distribution of the conduction data (e.g., with a Zero-centered 1 Dimensional Histogram Equalization (ZHE). As an example, the brain state embedder 612 can include an autoencoder, a decoder, and a plurality of adversarial training regularizers. In this example, the brain state embedder 612 can digest raw conduction data from the previously unseen patient and accommodate random channel order permutations of the conduction data from the plurality of recording electrodes via a cross-attention block. The cross-attention block can output at least two attention heads and the brain state embedder 612 exclude samples near the at least two attention heads with a buffered masking technique while allow future information to be embedded. The brain state embedder 612 can then use the autoencoder to blend a plurality of features (of the processed conduction data) from the at least two attention heads to preserve probabilistic embeddings, promote natural clustering, and prevent brain-state latent collapse. The autoencoder can be trained with an adversarial patient classifier and/or a discriminator. The brain state embedder 612 can take the conduction data and embed it into a common latent space, which can be an interpretable, well clustered, smooth latent space of blended brain-states across humans.

The brain state predictor 614 can predict a current brain state (e.g., with a given propensity) and/or at least one predicted future brain state in the form of embedded conduction data in the common latent space and/or embedded predicted future conduction data in the common latent space (e.g., an event). As an example, the brain state predictor 614 can include a variational autoencoder and a transformer. As an example, the brain state predictor 614 can resample outputs of the brain state embedder by feeding the outputs through a variational auto encoder to reduce redundant BSE embedding space into the number of dimensions. The brain state predictor 614 can transform the reduced outputs of the brain state embedder with context windows into predicted embedded future conduction data. And then, the brain state predictor 614 can project a brain-state trajectory based on the predicted embedded future conduction data the brain state trajectory can be the predicted future brain state, which can include a likelihood (e.g., propensity) of the prediction being true.

The brain state visualizer 616 can reduce and organize the conduction data in the common latent space and/or the at least one predicted future embedded conduction data in the common latent space into two dimension. The brain state visualizer 616 can then visualize, via the display and/or output device 112, at least one two dimensional (2D) topology map of a current brain state of the previously unseen patient and/or a predicted future brain state of the previously unseen patient. As an example, the brain state visualizer 616 can include a variational autoencoder and Kohonen Self-Organizing Map (SOM). In this instance, the brain state visualizer 616 can reduce the number of dimensions of the embedded conduction data and the predicted embedded future conduction data from the number of dimensions to a reduced number of dimensions and then organize the reduced dimension embedded conduction data and reduced dimension embedded future conduction data with the Kohonen SOM. The Kohonen SOM can be trained with data smoothed with 64 second windows and a stride of 16 seconds. As an example, the Kohonen SOM can be based on a hexagonal geometry and wrapped vertically and/or horizontally into a toroid configuration. In some instances, the brain state visualizer 616 can present the reduced dimension embedded conduction data and reduced dimension embedded future conduction data as the plurality of 2D topology maps of a current brain state of the previously unseen patient and a predicted future brain state of the previously unseen patient. The brain state model 610 can, in some instances make a determination and/or a prediction that a condition and/or a change in the condition is indicated based on the brain states, their likelihoods, and/or the 2D topology maps (e.g., based on locations, feature sizes, feature shapes, feature colors, etc.). In some instances, when an indication happens the system (e.g., 150) can provide an alert (e.g., audio, visual, tactile) to the patient, a medical professional, and/or a health record. In other instances, when an indication happens, then the system (e.g., 150) can control a treatment device as described in greater detail above.

### VII. Methods 2

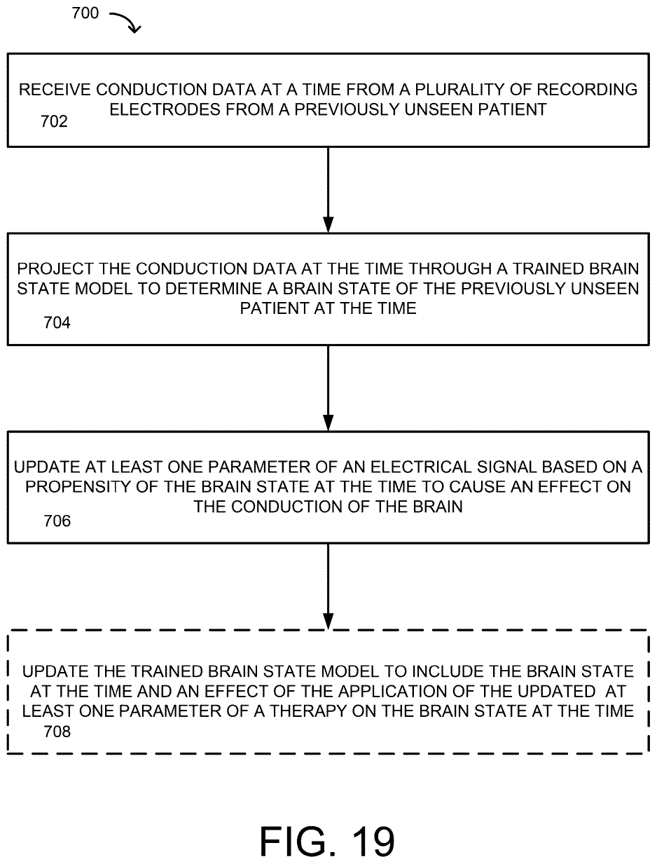

Another aspect of the present disclosure can include methods 700, 800, 900, and 1000 (FIGS. 19-22) for training and use of a (generalized) trained brain state model. The methods 700, 800, 900, and 1000 can be executed using the system 150 (shown in FIG. 1 and modified by FIGS. 2, 3, and 18). It should be understood that system 150 includes one or more recording electrode(s) that record conduction data from an area of at least part of the brain, a controller that receives the conduction data, implements a model of brain state to process the conduction data and determine a likelihood of the brain state contributing to a brain condition, and (if necessary) output a change of one or more parameters of a stimulation, a generator that receives the output and changes the one or more parameters and generates the stimulation, and one or more stimulating electrode(s) to deliver the stimulation to another at least part of the brain (which may be the same and/or different than where the conduction data is recorded).

For purposes of simplicity, the methods 700, 800, 900, and 1000 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 700, 800, 900, and 1000, nor are methods 700, 800, 900, and 1000 limited to the illustrated aspects. It should also be understood that any details described within this application can be applicable to both systems and methods.

The method 700 (FIG. 19) is for treating a previously unseen patient using a trained brain state model. At 702, conduction data can be received at a time from a plurality of recording electrodes (e.g., recording electrode(s) 102) from the previously unseen patient. The data can include a recording (e.g., of electrical signals) from at least a portion of the previously unseen patient's brain. For example, the conduction data at the time can be filtered into multi-dimensional time series data, equalized using a zero-centered one-dimensional histogram equalization to form equalized time series data, and then embedded into a multi-dimensional latent space using an asymmetric recurrent variational autoencoder to form the brain state data at the time. At 704, the conduction data can be projected at the time through a trained brain state model to determine a brain state of the previously unseen patient. It should be noted that the trained brain state model can be constructed without training data from the previously unseen patient. For example, the trained brain state model can be constructed by capturing blended brain-states across a plurality of patients, which can enable brain-state trajectory prediction for the previously unseen patient. In the example, a dimensionality of the brain state data at the time can be reduced and clustered at the time before one or more brain state groupings are identified that the brain state data corresponds to in the trained brain state model. The brain state data at the time can be reduced with a pairwise controlled manifold approximation and projection (PaCMAP) into at least one lower dimension of data and then clustered with a Hierarchical Density Based Spatial Clustering of Applications of Noise (HDBSCAN).

At 706, at least one parameter of an electrical signal can be updated based on a propensity of the brain state at the time (e.g., sliding scale from high propensity to low propensity) to cause an effect on the conduction of the brain. The propensity can be the likelihood that the determined brain state is correct. The brain state can be indicative of a condition (e.g., a neurological pathology like epilepsy). When a high propensity (e.g., 50% or greater, 60% or greater, 70% or greater, 80% or greater, 90% or greater, or the like likelihood of the brain state being correct) is indicated, then a generator can be alerted to generate (or update) a therapy (e.g., an electrical signal) to a stimulation device to treat the portion of the brain and/or another portion of the brain. For example, the at least one parameter and the effect of the condition are based on the blended brain-states across the plurality of patients and/or the conduction data at the time. At 708, in some instances, the trained brain state model can be updated to include the brain state at the time and an effect of the application of the updated at least one parameter of the stimulation (e.g., an electrical signal) on the bran state at the time to become at least partially patient-specific.

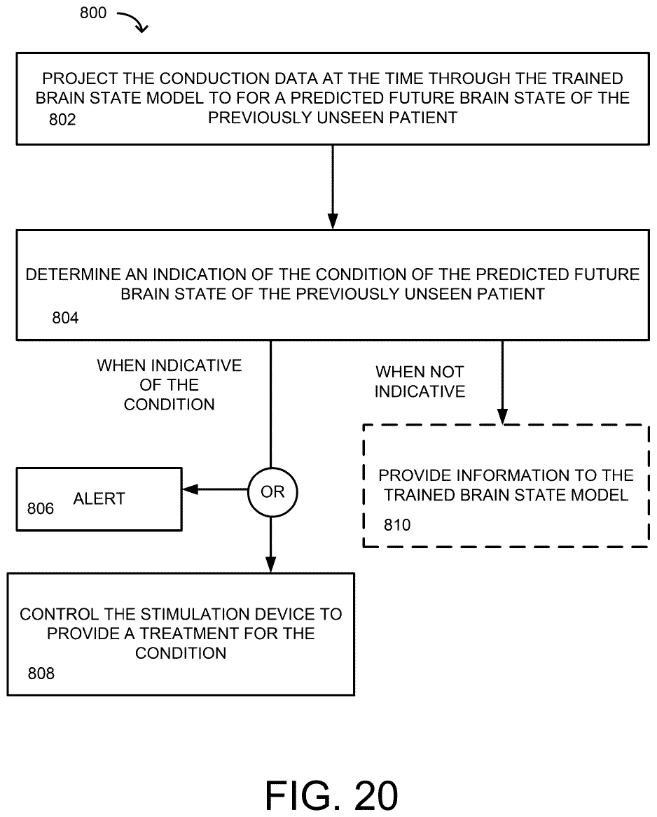

As shown in FIG. 20, the method 800 is for treating and/or preventing a change in a condition in a previously unseen patient. At 802, the conduction data at the time can be projected through the trained brain state model to predict a future brain state of the previously unseen patient. The predicted future brain state of the previously unseen patient can, in some instance, indicate a condition (e.g., epilepsy) and/or a change in a condition of the patient (e.g., seizure onset) At 804, an indication of the condition of the predicted future brain state can be determined (e.g., based on at least the visualization of the predicted future brain state output by the brain state visualizer and, optionally shown on the display/output device (e.g., 112), When the predicted future brain state is determined to be indicative of the condition (e.g., high risk) and/or a change in the condition, at 806 an alert can be generated (e.g., audibly, visual, and/or tactile) and sent to the patient, an associated medical professional, and/or an associated health record. The alert can include, for instance, the condition, the change in the condition, a suggested course of treatment, a timing of when the condition and/or the change in the condition is likely to occur, the strength of the indication, or the like. Additionally and/or alternatively, at 808, when the predicted future brain state is indicative of the condition and/or a change in the condition, then a stimulation device (e.g., in communication with controller 106, such as generator 104 and stimulating electrode(s)/device(s) 114) can be controlled to provide a treatment and/or preventative measure for the condition and/or the change in the condition to the patient. The treatment and/or preventative measure can be, but is not limited to, electrical stimulation, heat therapy, light therapy, cold therapy, pharmaceutical release, or the like. At 810, when the predicted future brain state is considered not indicative (e.g., low risk) of the condition and/or a change in the condition, then in some instances, the information can be provided to the trained brain state model (but in other instances, nothing may occur). It should be understood that similar steps can be taken for a currently occurring brain state determined by the brain state model as well.

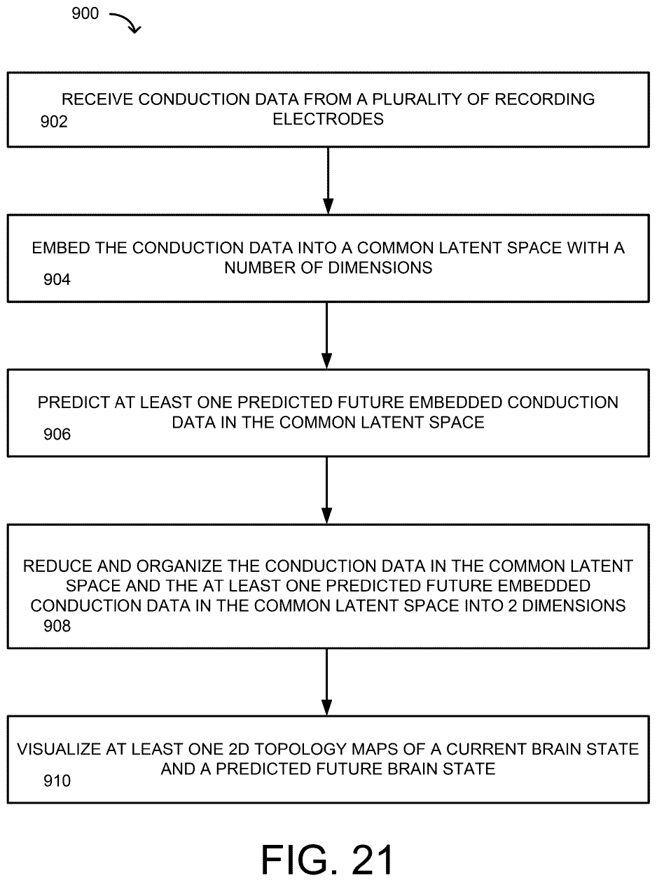

The method 900, shown in FIG. 21, is for visualizing at least one 2D topology map of a current brain state and/or a predicted future brain state of a previously unseen patient. At 902, conduction data can be received by the brain state model from a plurality of recording electrodes (e.g., recording electrical signals of a brain of a previously unseen patient, as described in detail above). At 904, the conduction data can be embedded into a common latent space with a number of dimensions (e.g., 1024) (as described in more detail above with respect to the brain state embedder). The conduction data in the common latent space can be projected through the brain state predictor (as described in more detail above) to predict the current brain state and/or the future brain state of patient based on the conduction data At 906, the conduction data (formed into the current and future brain state projections) can be reduced and organized (e.g., by the brain state visualizer) in the common latent space from 1024 dimensions to 8 dimensions and can then be further reduced from 8 dimensions into 2 dimensions, which can be understood by a human. At 910, at least one 2D topology map of the current brain state and/or the future brain state of the patient can be visualized (e.g., on a display such as display 112). The 2D topology map can include color indications of peaks and valleys indicative of various brain state information. In some instances, these 2D topology maps can be further analyzed to determine indications of conditions and/or changes in conditions of the patient as discussed above with respect to at least FIG. 20.

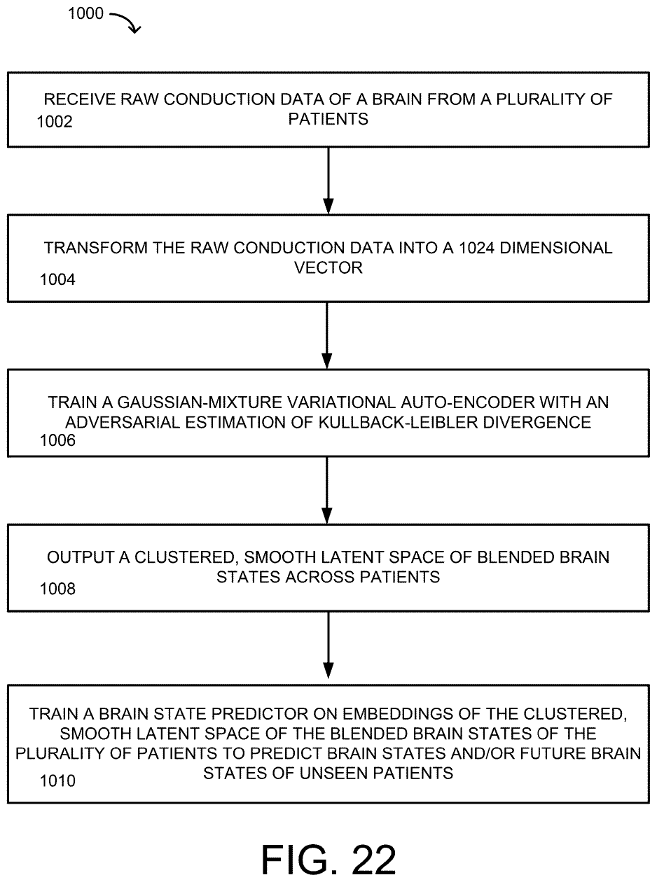

As shown in FIG. 22, the method 1000 is for training a brain state predictor to predict brain states and/or future brain states of previously unseen patients. At 1002, raw conduction data from a plurality of patients can be received (e.g., historical conduction data, saved from memory and/or from active recording electrode(s)). At 1004, the raw conduction data can be transformed into a 1024 dimensional vector. The raw data can be randomly ordered and padded with zeros up to 256 channels (e.g., for up to 256 recording electrodes) for every forward pass of the conduction data from the plurality of patients, by the system, through the trained brain state model. The conduction data (raw and/or randomly ordered and padded) can also be randomly frameshifted to create model capable of generalized determinations of current brain states and prediction of future brain states of patient's not previously seen by the model. At 1006, within the brain state model a Gaussian-mixture variational auto-encoder with an adversarial estimation of a Kullbuck-Leiber divergence can be trained with the randomized data. And at 1008, a clustered, smooth latent space of blended brain states across patients can be output within the model. At 1010, a brain-state predictor can be trained on embeddings of the clustered, smooth latent space of the blended brain states of the plurality of patients to predict brain states and/or future brain states of previously unseen patients.

### VIII. Experimental 2

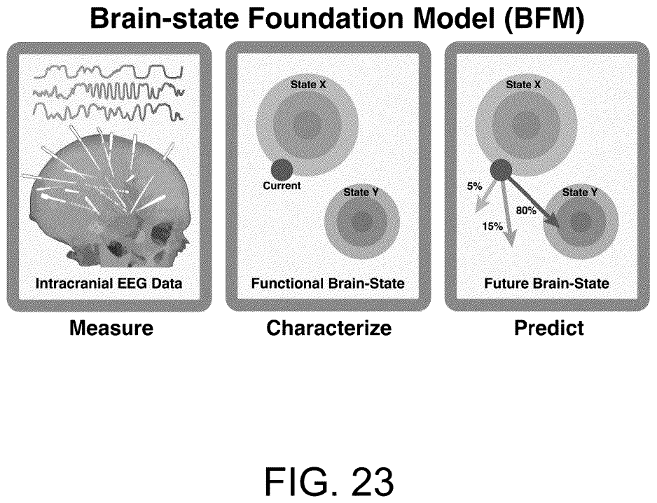

The following experiment shows a Brain-State Foundation Model (BFM) (FIG. 23), also referred to as a Brain-State Model (BSM), that can enable cross-subject brain state generalization. The goal of the BFM framework was to maximally extract biologically-relevant information from iEEG recordings to characterize the current state of a brain and predict future states. This paradigm was designed for direct applicability to adaptive neuromodulation for neurologic and psychiatric diseases. The term BFM was coined to describe the generalized foundation model and its use to drive brain-state characterization, prediction, and modulation. The BFM can enable one or more person's brain-states to inform the brain-states of others. And most importantly, obfuscate the need to re-train an entire model from scratch for each subject.

### 1. The BFM

The BFM 1) has generalizable properties, 2) allows interpretation of brain-state space, and 3) shows applicability to neuromodulation.

Closed-loop adaptive neuromodulation can be used for the treatment of neurologic and psychiatric diseases, the BFM was developed with this particular use case in mind. The simplest question about using an BFM to steer neuromodulation is that of driving “away from bad” states or “toward good” states-“Diseased Brain-State Postulates” are shown in Table 1. These postulates outline the interpretation of brain-states with the goal of neuromodulating a brain experiencing diseased states. Of particular importance is that of postulate 3-simply stated, without observing “good” states in a cohort, how can we know “where” in brain-state space to modulate the brain? If the only people receiving iEEG recordings are those with clinically-defined neurologic or psychiatric diseases, how can we obtain high quality physiologic data of healthy brain states? We believe that this is an intractable problem with ethical barriers that will not likely be overcome. Thus, this study focuses on the concept of neuromodulating “away from bad” states. Much work is to be done to make such a system a reality, but this may be possible with the fundamental insights into the characterization of abstracted brain-states with the BFM.

TABLE 1
Diseased Brain-State Postulates with Definitions and Implications
used to guide interpretation of Brain-State Model embedding spaces.
Diseased Brain-State Postulates

Definitions:
Embedding space: The smooth and regularized multidimensional space of possible
brain-states constructed from previously observed phenomena (e.g., intracranial
electroencephalography). The topology of the high-dimensional embedding space may be
visualized with dimensionality reduction techniques onto a two-dimensional map.
Brain-state/embedding: The exact mapped location within the embedding space.
Brain-state is synonymous with “embedding” in this context.
Embedding distance: The mathematical distance between two brain-states in the
high-dimensional latent space, as defined by metrics like Euclidean, Manhattan, or
angular distance. The higher the embedding distance, the more “different” the brain
states.
Brain-state trajectory: The sequential path of brain-states occupied through the
embedding space for a given epoch of time.

| | |
|---|---|
| Postulate 1 | No two brain-states antecedent to disease activity are identical. |

[Characterization] Exact embedding space occupied prior to previous clinically labeled disease
events (e.g., seizure, binge-eating, depressive episode) only serves as approximate landmark for
future events. Clinically-derived labels may conflate distinct brain states. Therefore, embedding
space construction benefits from a purely unsupervised/self-supervised model to allow unlabeled
brain-states to aggregate in a meaningful manifold.

| | |
|---|---|
| Postulate 2 | Temporal evolution of brain-states toward diseased activity can vary. |

[Prediction] Not all brain-states adjacent to a previously verified pre-disease state will evolve
identically - there is inherent stochasticity that is difficult to model. Brain-state prediction models
must accommodate this probabilistic nature.

| | |
|---|---|
| Postulate 3 | Brain-states “far” from observed diseased states are ill-defined. |

[Neuromodulation] In a subject cohort currently experiencing diseased brain-state activity, few
to no “stable” brain states far from disease activity are observed and mapped. Therefore, a
theoretical optimal brain-state target for closed-loop neuromodulation may not be readily defined
from previously observable brain states. Moreover, there is likely a constellation of potentially
stable brain states far from clinically significant disease activity.

The BFM is a first generalized brain-state model established according to the postulates

### 2. Methods

All model development was conducted in PyTorch. To develop a BFM, the choice of observable phenomenon was paramount to maximize potential information acquisition and generalization to unseen subjects. Many neurophysiological activity measurement techniques exist at various scales of resolution. For example, in increasing order of abstraction, there is ex vivo patch clamping, in vivo single unit microelectrode recordings, local field potentials (LFP) from intracranial macroelectrodes, neural circuit dynamics/connectivity between LFPs, and whole brain acquisition techniques like functional magnetic resonance imaging. For this study intracranial electroencephalography (iEEG) depth electrode recordings from humans undergoing clinical workup for drug-resistant epilepsy was chosen. The iEEG data offered the highest temporal resolution with minimal artifact, albeit sparse spatial sampling, and are recorded for days to weeks continuously per subject with increasingly common acquisition at integrated epilepsy centers.

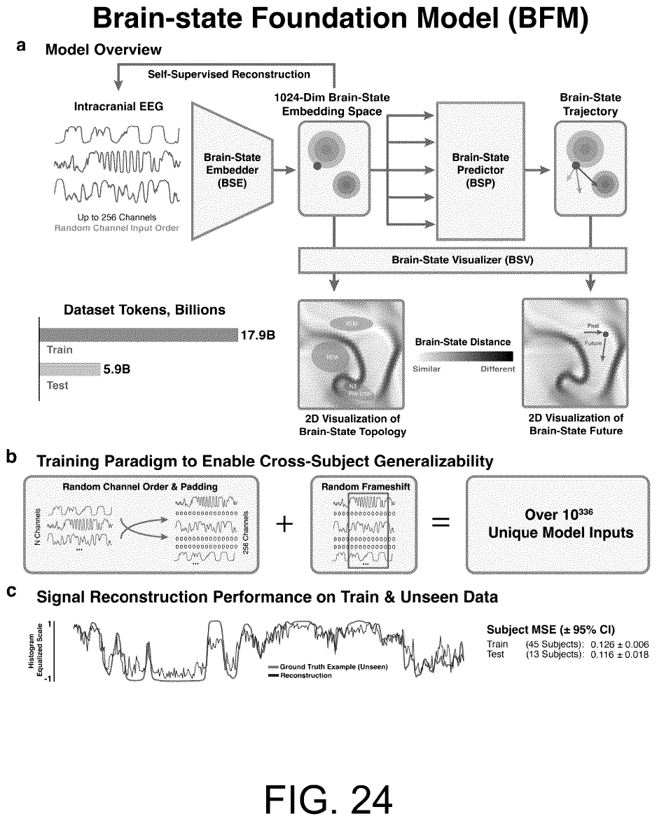

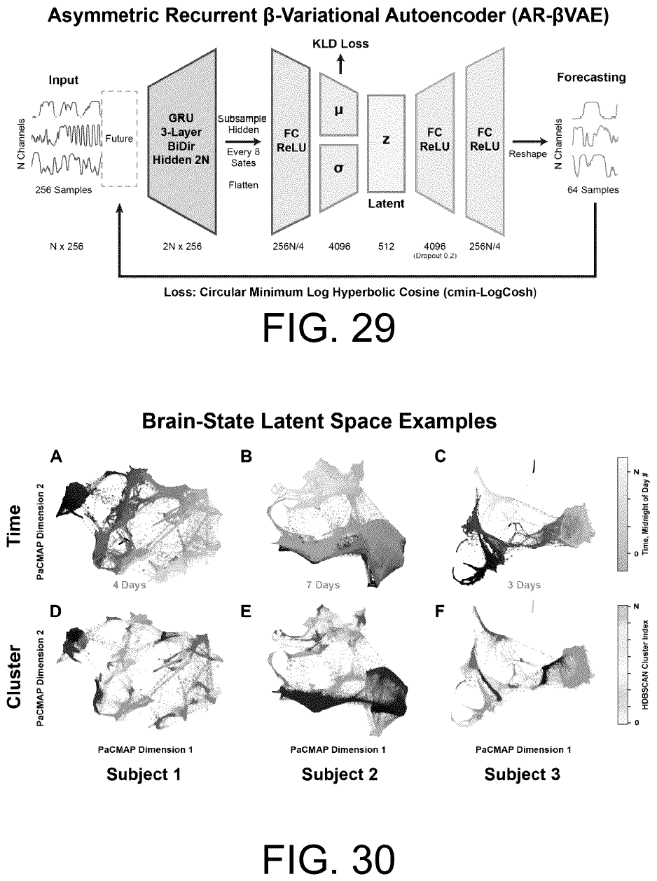

The next design decision was how to embed iEEG data into a meaningful high dimensional brain-state space. We opted for a transformer-based design with a highly randomized training paradigm to augment our 405 days (17.9 billion tokens) of iEEG recordings into virtually infinite input combinations (FIG. 24, elements a-b). The result is a pretrained BFM that is capable of ingesting unseen subjects' iEEG data and projecting their brain-state onto an interpretable manifold and accurately predicting future brain-states. A particularly powerful emergent property of the model is that the BFM is agnostic to input channel order, and robust to varying iEEG implant schemes (FIG. 24, element c). Additional information regarding the BFM is shown with relation to FIGS. 29-32.

FIG. 24 shows an overview of how data flows through the model and results of generalized raw signal reconstruction. a) iEEG data is first embedded into a common latent space with the Brain-State Embedder (BSE). Next, future embeddings (i.e. tokens) are predicted with the Brain-State Predictor (BSP). The embedding spaces of the BSE and BSP are 1024 dimensions. Thus, to interpret this high-dimensional space, the Brain-State Visualizer (BSV) brings the dimensions down to two and organizes brain states with a Kohonen Self-Organizing Map (SOM). b) To maximize the model's ability to generalize to unseen subject data, a heavily randomized training paradigm was used. Specifically, for every forward pass through the model, all subject's channels were randomly ordered and randomly padded with zeros up to 256 channels. Further, a random frameshift was applied that takes the 17.9 B training tokens into virtually infinite input variations into the model. c) The result of this generalized model architecture and training paradigm is indistinguishable reconstruction performance on raw iEEG data reconstruction between training data and unseen test data. MSE=mean squared error.

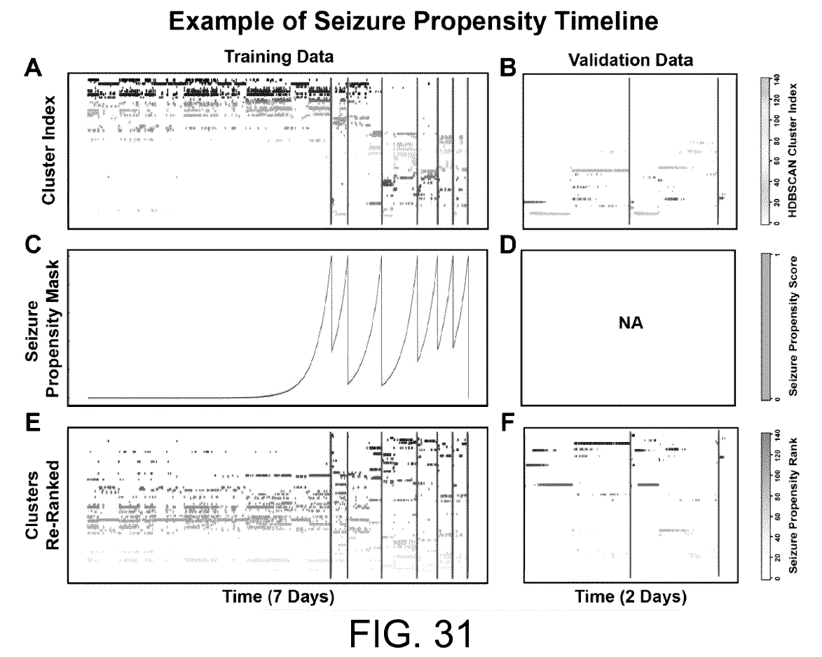

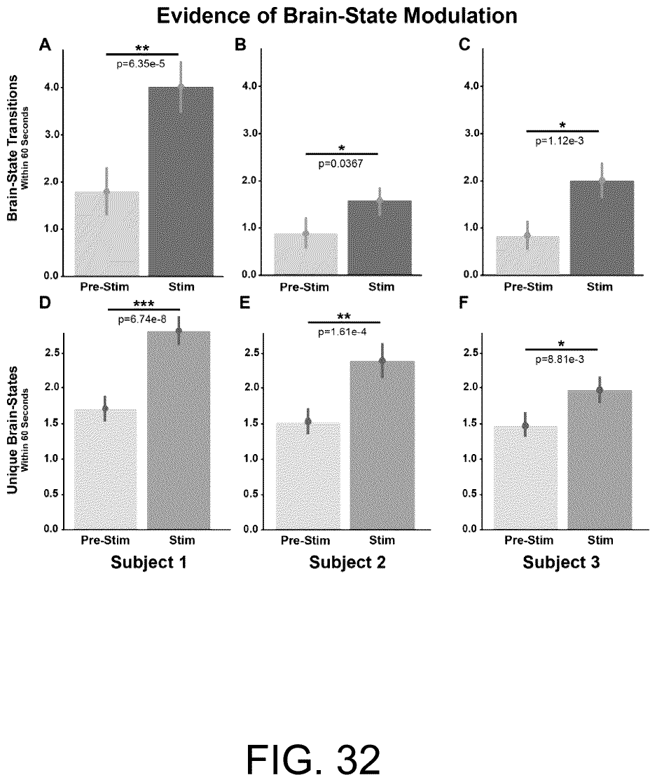

While FIGS. 29-32 shows additional details related to the BFM and the creation and testing of the BFM. FIG. 29 shows an example of a custom patient-specific model for brain-state embedding. Raw iEEG data is input, and future raw iEEG data is predicted through an asymmetric recurrent beta variational autoencoder. This model served as the first step toward the generalized Brain-state Foundation Modeling (BFM) presented in this study. FIG. 30 shows examples of manifold reduction of patient-specific brain-states for three different subjects. The top row outlines the brain-state progression over time (elements a-c), whereas the bottom row outlines the hierarchical clustering of brain-states (elements d-f). FIG. 31 shows example of using brain-state clusters to predict the likelihood of a patient experiencing a seizure. An entire hospital stay is outlined for this subject with training data to the left, and validation data on the right column. FIG. 31, element a demonstrates the natural cluster assignments in relation to ictal events (red vertical bars). FIG. 31, element b shows a quantification of seizure propensity based on likelihood that each cluster will experience a seizure. FIG. 31, element c shows a re-classification of cluster indexing based on seizure propensity. Finally, FIG. 32 shows examples of low-energy single-pulse electrical stimulation (SPES) effects on brain-states. 1 Hz 3 mA biphasic 300 microsecond balanced square wave SPES was significantly more likely to promote brain-state transitions (FIG. 32, elements a-c), and more likely to promote unique brain-state occupation compared to resting state epochs (FIG. 32, elements d-f).

### 2.1 Preprocessing

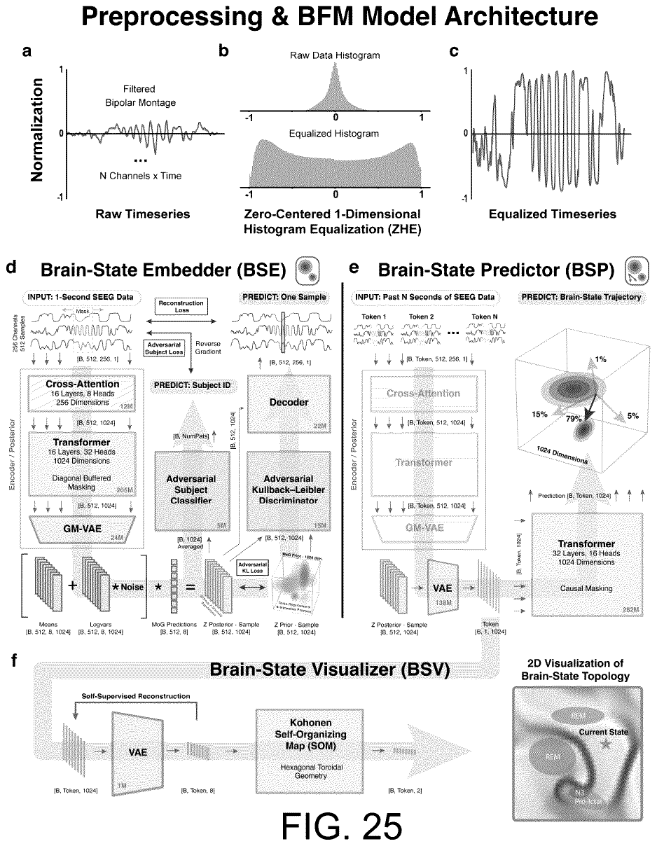

The training dataset consisted of 45 subjects with iEEG implantation, with a range of 88-190 channels per subject (mean 134.2, std: 26.4). The completely withheld test dataset consisted of 13 subjects with a range of 52-177 channels per subject (mean 127.5, std: 37.3). Channels that were out of the brain parenchyma on post-implantation imaging, and clear artifactual channels were excluded. The raw iEEG data were subsampled to 512 Hz and referenced with an adjacent bipolar montage and filtered with infinite impulse response zero-phase digital filters using pass windows of 1-59, 61-119, 121-179 Hz. To maximize generalization, all subject's data must be properly scaled into a common range. Further, machine learning paradigms benefit from an equally distributed range of values. Thus, we developed a custom histogram equalization scheme called Zero-Centered 1-Dimensional Histogram Equalization (ZHE) (FIG. 25, elements a-c).

The ZHE is conducted by reading in the first 24 hours of iEEG recordings for each subject, then parsing the signal into positive and negative values. Each positive/negative domain is processed separately. Each domain is histogram equalized using 10,000 bins. Then each equalized bin is saved as a linear function to be applied to the remainder of that subject's data (FIG. 25 element c). The utilization of only the first 24 hours of a subject's data is what results in an evenly distributed but not perfectly uniform distribution as exemplified in FIG. 25, element b. This paradigm allows for any new data from that subject to be equalized with an existing scheme without need for an entirely new equalization calculation.

FIG. 25 shows the detailed architecture of the BFM, also referred to as the brain state model. FIG. 25, elements a-c show that the raw iEEG data is first referenced with an adjacent bipolar montage and filtered with infinite impulse response zero-phase digital filters using pass windows of 1-59, 61-119, 121-179 Hz. Next, a custom equalization scheme is used that preserves zero-centering and optimizes the data distribution for machine learning, coined Zero-Centered 1-Dimensional Histogram Equalization (ZHE), and is defined here in the Methods section. FIG. 25, element d shows the Brain-State Embedder (BSE) consists of an encoder (a.k.a posterior), decoder, and adversarial training regularizers. For further details discussed below. FIG. 25, element e shows the Brain-State Predictor (BSP) utilizes the posterior from the BSE. The resampled outputs of the posterior are first fed through a vanilla VAE to reduce the 524,288 redundant BSE embedding space into 1024 dimensions. Next, each token (representing 1 second of iEEG data) is fed into the BSP transformer with context windows of 32 seconds and trained in a traditional manner with causal masking of attention, discussed more below. FIG. 25, element f shows, finally, in order to visualize the BSP embedding space, a Brain-State Visualizer (BSV) that is trained to further reduce the 1024 dimensions to 8, then feeds this into a Kohonen Self-Organizing Map (SOM). The SOM is trained on all data smoothed with 64 second windows and a stride of 16 seconds. The SOM aims to properly represent the high-dimensional manifold topology in 2-dimensions.

### 2.2 Brain-State Foundation Model (BFM)

The detailed architecture of the BFM can be seen in FIG. 25, elements d-f with data tensor sizes as information flows through the forward pass of the model. The model is split into three distinct modules, the Brain-State Embedder (BSE), the Brain-State Predictor (BSP), and the Brain-State Visualizer (BSV).

### 2.2.1 Brain-State Embedder (BSE)

There are key components of the BSE architecture and training paradigm that enable the powerful generalization of the BFM to unseen subject data. Specifically, the use of a Gaussian-Mixture Variational Autoencoder (GM-VAE) in the BSE to simultaneously promote unique brain-states while also melding subjects' brain-states into a smooth and complete high-dimensional data manifold in 1024 dimensional embedding space. Next, the randomized training paradigm outlined in FIG. 24, element b that augments the data input space to virtually infinite permutations and ensures the model does not overfit to certain channel order or temporal event locking. Training stopping criteria was evidence lower bound (ELBO). A powerful result of this paradigm is that the GM-VAE is capable of reconstructing subject data regardless of input order, implantation scheme, or even if the model has seen that subject's data before. This suggests that the BSE has developed a generalized framework for the fundamental biophysical reality and statistical modeling of iEEG data as a whole.

The BSE (FIG. 25, element d) consists of an encoder (a.k.a posterior), decoder, and adversarial training regularizers. The first layer of the posterior is a cross-attention block that extracts channel-to-channel motifs. Next, a transformer block is used to extract large scale patterns across time. Importantly, the transformer is diagonally masked with a 16-sample buffer on either side to allow imputation of reconstructed data blinded to 32 samples total. The final layer of the posterior is an 8-component GM-VAE to promote distinct brain-states, but also merge subjects' brain-states into a smooth and complete manifold in high-dimensional space. As with all VAE paradigms, the output of the GM-VAE is re-parameterized and passed through the decoder to reconstruct a single data point at a time. Next in the forward pass, there are two adversarial components: 1) an adversarial subject classifier with a reversal of gradients during backpropagation to encourage the model to place subjects into the latent space agnostic to subject identification, 2) the Kullback-Leibler calculation for an 8-component GM-VAE is computationally prohibited with Monte-Carlo simulations, thus an adversarial approach is used to train the model to fool a discriminator against samples from the GM 8-component prior. The BSE is trained in isolation to properly balance the delicate adversarial relationship with the intricate GM-VAE regularization.

### 2.2.2 Brain-State Predictor (BSP)

Next, the BSP is trained in conjunction with the BSV (described below). The pre trained BSE is utilized in inference mode and random channel-order inputs are once again fed into the posterior of the BSE. The outputs of the BSE posterior are then directly fed into the BSP architecture described in FIG. 25, element e (i.e. the adversarial and decoder components of the BSE are no longer utilized). The first stage of the BSP is a vanilla VAE that takes the redundant 512×10²⁴ dimensional space of the BSE down to 1×10²⁴ dimension tokens where each token represents one second of iEEG data. The BSP then consists of a transformer that is traditionally trained on causal masking of context windows of 32 seconds/tokens of iEEG data to predict next tokens. In parallel, the 1×10²⁴ dimensional space is simultaneously detached from the computational graph and forward passed through the BSV.

### 2.2.3 Brain-State Visualizer (BSV) with Kohonen Self-Organizing Map (SOM)

The BSV training does not affect the BSP training and is simply a tangential model arm to further reduce the dimensionality of the embedding space for use in clinical interpretation of brain-states. The BSV is a vanilla VAE that takes the dimensions from 1024 to 8. The outputs of the BSV are collected for the entire dataset and used to construct a hexagonal geometry SOM with toroidal wrapping. The SOM is a 64×64 hexagonal grid that wraps left-right and up-down (i.e. a toroid). The entire training and test set are used to construct this visualization. The test set was never used in the BSE, BSP, or BSV training, but it is important to use it in the SOM visualization to allow adjacent locations along the high-dimensional manifold to not be overly simplified by the linear SOM algorithm. The U-Matrix topology best characterizes the high-dimensional manifolds as seen in FIG. 25, elements a-b. Areas of high U-Matrix value correlate to brain-states that are very distant from the nearest neighbor in the hexagonal grid. Thus, the black ridges in the U-Matrix act as mountain barriers that are unlikely to be traversed by a person's brain-state. Post-hoc clinical labels can then be overlaid to interpret the brain-state topology.

### 3. Results

The trained BFM, with the stopping criteria of ELBO, demonstrated a mean-squared error (MSE) value in 1000 random training data epochs (45 subjects) of 0.126+/−0.006 and a validation MSE in 1000 completely withheld test data epochs (13 subjects withheld) of 0.116+/−0.018. An example of the predicted iEEG waveform is seen in FIG. 24, element c). The BFM generalizes to unseen data despite significant subject heterogeneity in iEEG implantation scheme, seizure semiology, and presumed fundamental differences in brain characteristics. This is promising evidence for the generalization of the BFM.

### 3.1 the BFM can Characterize Interpretable, Clinically-Meaningful, and Generalized Human Brain-States

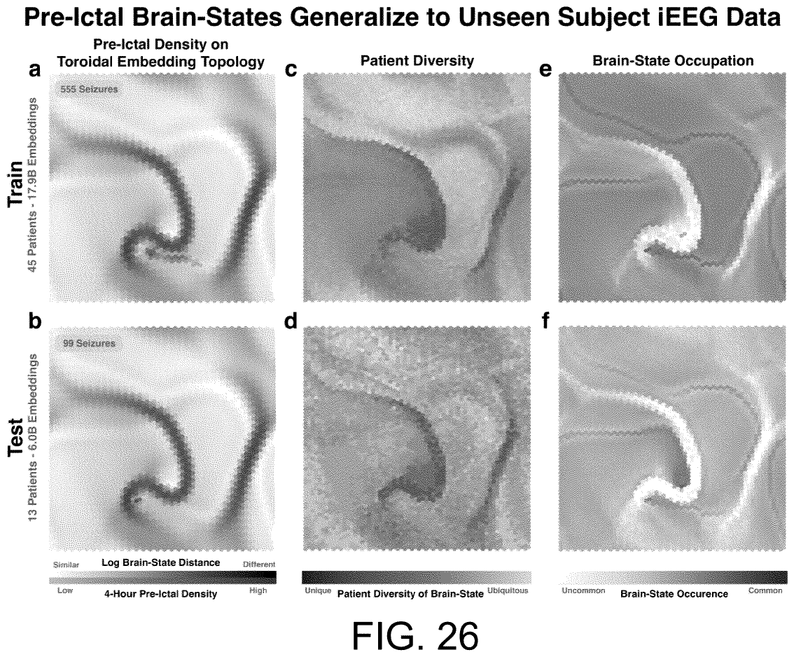

The BFM architecture operates in 1024-dimensional latent space. Thus, the Brain-State Visualizer (BSV) has been trained in parallel to help interpret the lower dimensional manifold that exists within the high-dimensional space (FIG. 25, element f). Specifically, a Kohonen Self-Organizing Map (SOM) was built to aggregate similar brain-states. It is important to note that there was no element of supervision in the training process—i.e. all levels of the BFM architecture are unsupervised or self-supervised, including the SOM creation. The SOM of the training and completely withheld 230 test datasets can be seen in FIG. 26.

In summary FIG. 26 shows post-hoc interpretation of the BFM embedding space. The BFM model was trained on 17.9 B unique input tokens, which comprises over 405 days' worth of iEEG data from 45 subjects. The model was then tested on 6.0 B tokens which comprises 135 days worth of iEEG data from 13 subjects. Importantly, the test data were completely withheld from model training. The visualizations shown in the figure are dimensionality reductions of the 1024 latent space into 2D with the help of the BSV. As noted, the BSV consists of a variational autoencoder and a Kohonen Self-Organizing Map (SOM). The SOM is based on a hexagonal geometry and is wrapped vertically and horizontally into a toroid configuration (i.e. going off the right of the map wraps around to the left and similarly for up and down). The top row of plots represent the training data, and the bottom row represents completely withheld test data.

FIG. 26, element a shows a white and gray visualization of the SOM that is known as the U-Matrix and represents the distance each brain state is from its neighbors. A darker color on the U-Matrix implies that the brain-state is very far from its neighbors on the native 1024 dimension manifold. Thus, black ridges can be thought of as mountain ranges that are difficult for the brain-state to navigate across. The U-Matrix is a static representation of the topology of the brain-state embedding space, and we can then add post-hoc labels to the U-Matrix to represent clinically important states. Here we have added a contour of 4 hours from known pre-ictal events thresholded at a density of 0.5. There were 555 seizures in the training data that were annotated by board-certified epileptologists. We defined preictal as 4 hours before each of these electroclinical seizures. We define areas included in this pre-ictal density and areas adjacent to these areas as “pro-ictal” to indicate high seizure risk states. FIG. 26, element b, shows a U-Matrix visualization that is the same as above, but now overlaid with the completely withheld test data 4-hour pre-ictal density onto the plot. As expected with a properly generalized model, the test pre-ictal density aligns with the region we have considered pro-ictal based on the training data.

The grayscale colors in FIG. 26, elements a-b represent the “U-Matrix” of the SOM where darker colors indicate brain-states that are further apart in the original 1024-dimensional space. Thus the dark ridges in FIG. 26, elements a-b are akin to peaks in brain-state topology that are unlikely for a brain to traverse. The SOM is of hexagonal geometry and wraps up-down and left-right into a toroidal surface which is flattened into 2D here. Clinically-relevant labels can then be overlaid post-hoc onto the SOM, as is done in FIG. 26, element a with the 4-hour pre-ictal density.

Specifically, the 555 epileptologist-defined electroclinical seizures represented across the train dataset subjects were taken and labeled and the 4-hour pre-ictal period was thresholded at 0.5 contour density. This region of the SOM was called “pro-ictal” because there are many instances of subjects visiting this area, but not immediately evolving into a seizure. This is the desired utility of the BFM—to aggregate functionally similar brain-states and sparsely label them with incomplete clinical information. Importantly, the pro-ictal region of the SOM generalizes to the completely withheld 13 subjects comprising 99 electroclinical seizures from the test dataset (FIG. 26, element b). To assess the ubiquity of the brain-states, FIG. 26 elements c-d demonstrate the proportion of patients that visit each brain state—the lighter (yellow in color) indicates states visited by every subject, where the darker (purple in color) represents states only visited by a few subjects. As expected, the states that are black on the U-Matrix (FIG. 26, elements a-b) are not visited by many subjects because they are rare states in general. Finally, FIG. 26, elements e-f represent how often a state is visited. This is different from plots FIG. 26, elements c-d in that a state may be considered diverse if every subject visits that state, but it still may be a very rare state. Thus, FIG. 26, element e-f showcase how common each state is with darker (blue in color) indicating more overall occupancy of that state throughout the entire dataset. As expected, the black ridges in the U-Matrix are rarely visited.

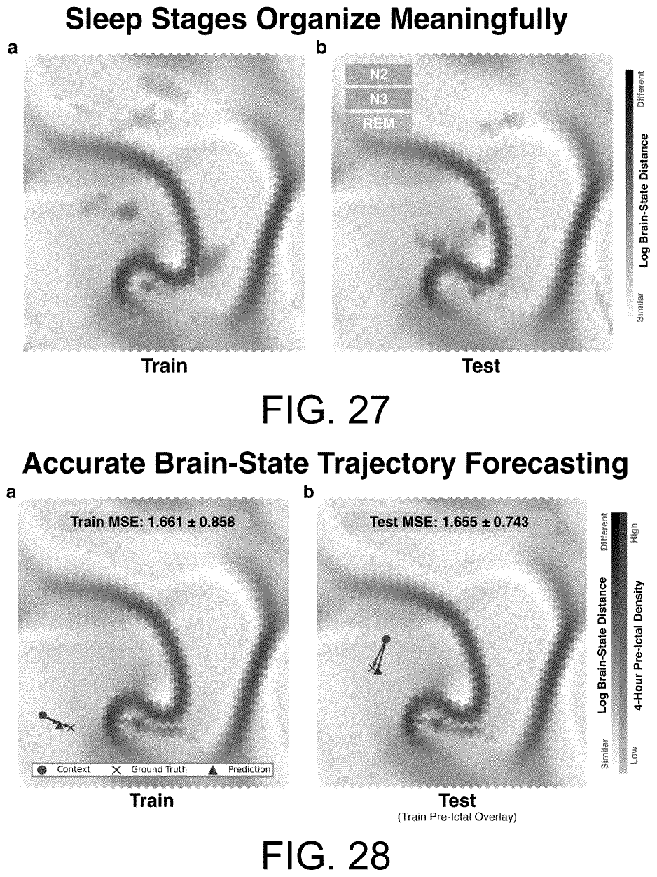

Although these data were gathered from people with drug-resistant epilepsy, the utility of the BFM is not restricted to peri-ictal brain-state delineation. To demonstrate the richness of brain-state information present in large-scale iEEG recordings has been overlaid with sleep stages as defined by the Montreal Neurological Institute (MNI) SleepSEEG algorithm to demonstrate the brain-state organization of sleep (FIG. 27). In FIG. 27 the sleep stages were generalized to unseen subject recordings. U-Matrix visualization with sleep stages overlaid (N2, N3, REM). Sleep stages were defined with Montreal Neurologic Institute (MNI) sleep staging algorithm SleepSEEG, thresholded at above 50% model confidence for 5 minutes, excluding N1 and excluding any night upon which a seizure occurred. FIG. 27, element a shows sleep stages for the training data, notably N3 sleep overlaps heavily with the pro-ictal brain-state locations, and REM sleep is very far across high U-Matrix ridges. This observation is in alignment with previous notions that N3 is a high risk state for seizures and REM is generally protective against seizure genesis. FIG. 27, element b shows test data generalizes well for N3 and REM sleep, but not for the more difficult to characterize N2 sleep.

Of clinical interest, N3 sleep organizes closely to pro-ictal epochs which is in alignment with previous work postulating the relatively high seizure generation risk of N3 sleep. Conversely, rapid eye movement (REM) sleep is across the U-Matrix “ridge” from the pro-ictal brain-state region and is thought to be protective against seizure generation. Interestingly, REM sleep is further divided into 2 distinct subgroups which suggests that this sleep stage was oversimplified by defining both as REM-which is perhaps explained by phasic vs. tonic REM. Overall, the BFM shows promise in defining a new generalizable state space upon which to test hypotheses and develop neuromodulation interfaces.

### 3.2 Future Brain-States can be Accurately Predicted on Unseen Human Data

Of particular interest is the advancement of adaptive neuromodulation paradigms to treat neurologic and psychiatric diseases. The utility of the BFM in this endeavor is predicated on the ability to predict future brain-states. As shown in FIG. 28, the BFM is able to accurately predict brain-state trajectories with comparable efficacy in the training and completely withheld test sets
the mean-squared error (MSE) of 100 predictions is displayed overlying a representative prediction for the training (FIG. 28, element a) and test (FIG. 28, element b) datasets. The ability to predict the future from a new subject's raw data that was not utilized in model training is a non-trivial contribution to the future of adaptive neuromodulation paradigms. FIG. 28 shows Brain-State trajectory mapping with BSP. A vital concept for possible neuromodulation paradigms using the BFM is the ability to predict the future brain-states. Of more significance is the ability to predict future brain-states for subjects not involved in the training of the model. Exemplified in FIG. 28 is the BFM's ability to predict future brain-states of subject's that it is has never seen before. In FIG. 28, element an example of trajectory prediction for the training data is shown and, in FIG. 28, element b, the test data. The training mean-squared error (MSE) of the future predictions is indistinguishable from the test data (1.661±0.858 95% CI vs. 1.655±0.743, respectively)

### 1.6 Related Work

A related, but distinct area of work is that of behavior prediction or clinical forecasting models like seizure prediction with EEG. The BFM was developed in direct response to the pitfalls of such models. Clinical prediction models based on physiologic data are dependent on supervised learning paradigms that leverage expert labeling of clinical data. This technique will always be limited by the quality of the label, the conflation of a single label to potentially distinct states (e.g. REM sleep in FIG. 28), and the confidence of pertinent or assumed negative labels (e.g. all the brain-states adjacent or coincident with the pro-ictal states, but were not labeled as such). The BFM paradigm does not make any assumptions about data labeling. Thus, the BFM serves as a foundation model for brain-state exploration and a source of potential utility in neuromodulation paradigms where a smooth, complete, and continuous manifold upon which to adaptively modulate is crucial.

From the above description, those skilled in the art will perceive improvements, changes, and modifications. Such improvements, changes and modifications are within the skill of one in the art and are intended to be covered by the appended claims.

The following is claimed:

1. A method for closed-loop neuromodulation to treat a condition of a brain of a previously unseen patient, the method comprising:
receiving, by a system comprising a processor, conduction data at a time from a plurality of recording electrodes in communication with the processor, wherein the plurality of recording electrode record conduction data from at least a portion of the brain of the previously unseen patient;
projecting, by the system, the conduction data at the time through a trained brain state model to determine a brain state of the previously unseen patient at the time, wherein the trained brain state model is constructed without training data from the previously unseen patient, wherein the trained brain state model is constructed by capturing blended brain-states across a plurality of patients and enables brain-state trajectory prediction for the previously unseen patient; and
updating, by the system, at least one parameter of an electrical signal based on a propensity of the brain state of the previously unseen patient at the time to cause an effect of the condition of the brain,
wherein, based on the propensity of the brain state, at least a generator generates the electrical signal and provides the electrical signal to at least one stimulation device that applies the electrical signal to at least another portion of the brain, wherein the at least one parameter and the effect of the condition are based on the blended brain-states across the plurality of patients and the conduction data at the time.

2. The method of claim 1, further comprising predicting a future brain state of the previously unseen patent by:
projecting, by the system, the conduction data at the time through the trained brain state model to form a predicted future brain state of the previously unseen patient;
determining, by the system, an indication of the condition of the predicted future brain state of the previously unseen patient;
when the predicted future brain state of the previously unseen patient is indicative of the condition:
alerting, by the system, at least one of the patient, a medical professional, and an electronic health record, and/or
controlling, by the system, stimulation device in communication with the system to provide a treatment for the condition.

3. The method of claim 1, wherein the condition of the brain is a neurological pathology.

4. The method of claim 3, wherein the neurological pathology is epilepsy.

5. The method of claim 1, wherein the conduction data at the time is filtered into multi-dimensional time series data, equalized using a zero-centered one-dimensional histogram equalization to form equalized time series data, and then embedded into a multi-dimensional latent space using an asymmetric recurrent variational autoencoder to form the brain state data at the time.

6. The method of claim 5, further comprising reducing a dimensionality of the brain state data at the time and clustering the reduced dimensionality brain state data at the time before one or more brain state groupings are identified that the brain state data corresponds to in the trained brain state model.

7. The method of claim 6, further comprising reducing the brain state data at the time with a pairwise controlled manifold approximation and projection (PaCMAP) into at least one lower dimension of data and then clustered with a Hierarchical Density Based Spatial Clustering of Applications of Noise (HDBSCAN).

8. A system comprising:
a plurality of recording electrodes configured to record conduction from at least a portion of a brain of a previously unseen patient;
an output device; and
at least one controller, in communication with the plurality of electrodes and the display device, each controller comprising:
a memory configured to store a trained brain state model and instructions, wherein the trained brain state model comprises a brain state embedder, a brain state predictor, and a brain state visualizer, and wherein the trained brain state model was not trained on the conduction data of the previously unseen patient, and
a processor configured to execute at least a portion of the instruction to:
receive by the brain state embedder, the conduction data from the plurality of recording electrodes;
embed, by the brain state embedder, the conduction data into a common latent space with a number of dimensions, wherein the brain state embedder forms outputs comprising at least embedded conduction data in the common latent space;
predict, by the brain state predictor, at least one predicted future embedded conduction data in the common latent space;
reduce and organize, by the brain state visualizer, the conduction data in the common latent space and the at least one predicted future embedded conduction data in the common latent space into two dimension; and
visualize, by the brain state visualizer via the output device, at least one two dimensional (2D) topology map of a current brain state of the previously unseen patient and/or a predicted future brain state of the previously unseen patient.

9. The system of claim 8, wherein the conduction data embedded in the common latent space comprises 1024 dimensions.

10. The system of claim 8, wherein the trained brain state model is trained to generalize to the conduction data of the previously unseen patient with a heavily randomized training paradigm comprising:
randomly ordering and padding with zeros up to a number of channels of a maximum number of the plurality of electrodes for every forward pass of known subject data through the trained brain state model, and
randomly frameshifting current and predicted brain state conduction data of the known subjects.

11. The system of claim 8, wherein the processor is further configured to execute the instructions to:
reference the conduction data received from the plurality of electrodes;
filter the conduction data with infinite impulse response zero-phase digital filters; and
optimize a data distribution of the conduction data.

12. The system of claim 8, wherein the brain state embedder comprises an autoencoder, a decoder, and a plurality of adversarial training regularizers.

13. The system of claim 12, wherein the processor is further configured to execute the instructions to:
digest raw conduction data from the previously unseen patient and accommodate random channel order permutations of the conduction data from the plurality of recording electrodes via a cross-attention block, and wherein the cross-attention block outputs at least two attention heads;
exclude samples near the at least two attention heads with a buffered masking technique while allow future information to be embedded;
blend a plurality of features, via the autoencoder, from the at least two attention heads to preserve probabilistic embeddings, promote natural clustering, and prevent brain-state latent collapse, wherein the autoencoder is trained with an adversarial patient classifier and/or a discriminator; and
output conduction data embedded in the common latent space, wherein the common latent space is an interpretable, well clustered, smooth latent space of blended brain-states across humans.

14. The system of claim 8, wherein the brain state predictor comprises a variational autoencoder and a transformer.

15. The system of claim 14, wherein the processor is further the processor is further configured to execute the instructions to:
resample outputs of the brain state embedder by feeding the outputs through a variational auto encoder to reduce redundant BSE embedding space into the number of dimensions;
transform the reduced outputs of the brain state embedder with context windows into predicted embedded future conduction data; and
project a brain-state trajectory based on the predicted embedded future conduction data.

16. The system of claim 8, wherein the brain state visualizer comprises a variational autoencoder and Kohonen Self-Organizing Map (SOM).

17. The system of claim 16, wherein the processor is further configured to execute the instructions to:
reduce the number of dimensions of the embedded conduction data and the predicted embedded future conduction data from the number of dimensions to a reduced number of dimensions;
organize the reduced dimension embedded conduction data and reduced dimension embedded future conduction data with the Kohonen SOM, wherein the Kohonen SOM is trained with data smoothed with 64 second windows and a stride of 16 seconds; and
represent the reduced dimension embedded conduction data and reduced dimension embedded future conduction data as the plurality of 2D topology maps of a current brain state of the previously unseen patient and a predicted future brain state of the previously unseen patient.

18. The system of claim 17, wherein the Kohonen SOM is based on a hexagonal geometry and is wrapped vertically and horizontally into a toroid configuration.

19. A method for training a brain state model to reconstruct conduction data from a previously unseen patient and encode brain-state embeddings into clinically meaningful clusters within a latent space, the method comprising:
receiving, by a system comprising a processor, raw conduction data of a brain from a plurality of patients;
transforming, by the system, the raw conduction data into a 1024 dimensional vector;
training, by the system, a Gaussian-mixture variational auto-encoder (GM-VAE) with an adversarial estimation of Kullback-Leibler divergence;
outputting, by the system, a clustered, smooth latent space of blended brain states across patients; and
training, by the system, a brain state predictor on embeddings of the clustered, smooth latent space of the blended brain states of the plurality of patients to predict future brain states.

20. The method of claim 19, further comprising:
randomly ordering and padding with zeros up to 256 channels for every forward pass of the conduction data from the plurality of patients, by the system, through the trained brain state model, and
randomly frameshifting, by the system, current and predicted brain state conduction data of the plurality of patients.
